(19) United States
(12) Patent Application Publication
(10) Pub. No.: US 2015/0203467 A1
(43) Pub. Date: Jul. 23, 2015

(54) METHOD FOR PRODUCING LINEAR AND/OR CYCLIC CARBONATE ESTERS

(71) Applicant: Bayer MaterialScience AG, Leverkusen (DE)

(72) Inventors: Thomas Ernst Müeller, Aachen (DE); Christoph Gürtler, Köhl (DE); Sait Elmas, Alsdorf (DE); Burkhard Köhler, Zierenberg (DE); Waltre Leitner, Aachen (DE); Markus Harrer, Marburg (DE); Jörg Sundermeyer, Marburg (DE)

(73) Assignee: Bayer MaterialScience AG, Leverkusen (DE)

(21) Appl. No.: 14/422,777

(22) PCT Filed: Aug. 19, 2013
(86) PCT No.: PCT/EP2013/067207

§ 371 Date: Feb. 20, 2015

(30) Foreign Application Priority Data
Aug. 23, 2012 (EP) 12181442.0

(51) Int. Cl. C07D 317/36; C07D 317/46; C08G 64/34

(52) U.S. Cl. CPC C07D 317/36; C08G 64/34; C07D 317/46

(57) ABSTRACT

The present invention relates to a method for producing linear and/or cyclic carbonate esters, involving the step of reacting an epoxide with carbon dioxide in the presence of a catalyser. The invention further relates to the use of special catalysers for reacting epoxides with carbon dioxide, special catalysers and special reaction products. The catalyser comprises a complex of a metal M or M-A with a ligand L, the metal M being present in an oxidation state of >=0, A standing for halide, carboxylate, phenolate, sulfonate, phosphonate, alkyl, alkoxy or amido, and the ligand L having the subsequent structure (Ia) or (Ib), wherein one or both of the OH groups shown in (Ia) and/or (Ib) can also be deprotonated.

The present invention relates to a method for preparing linear and/or cyclic carbonate esters comprising the step of reacting an epoxide with carbon dioxide in the presence of a catalyst. It further relates to the use of specific catalysts for the reaction of epoxides with carbon dioxide, specific catalysts and also specific reaction products.

The reaction of epoxides with carbon dioxide is of interest from several aspects. Firstly, this reaction in very general terms serves to fix CO₂, which is obtained, for example, in the purification of exhaust gases from incineration plants. Furthermore, linear and/or cyclic carbonates (carbonate esters) can be obtained. Linear carbonate esters are used as polymeric materials. Cyclic carbonate esters may be used, inter alia, as solvents, as electrolytes in batteries or as transesterification reagents. Chemical Communications 47(2011)141-163 gives an overview of the known catalysts for copolymerizing epoxides and CO₂.

In relation to another catalyst class, WO 2009/095164 A1 discloses a method for preparing urethanes by oxidative carbonylation of amino compounds in the presence of carbon monoxide, oxygen and organic compounds bearing hydroxyl groups. The carbonylation is carried out in the absence of halogen-containing promoters and in the presence of metal complex catalysts comprising neutral bidentate N-chelate ligands of the type (N˜N), two monoanionic N,O-chelate ligands of the general type (N˜O)⁻ or tetradentate dianionic chelate ligands (O˜N˜N˜O)²⁻.

JP 2003/342287 A discloses the preparation of linear carbonate esters by reacting an epoxide with carbon dioxide in the presence of a zinc complex comprising an electrically neutral bidentate ligand, for example 2,9-bis(2′,6′-dimethylphenyl)phenanthroline.

WO 2008/135337 A1 describes bis(hydroxyquinoline)-metal complexes and the use thereof as bleach catalysts.

With the catalyst systems currently used for copolymerizing epoxides with carbon dioxide, a technically applicable activity is only reached at elevated temperatures. The resulting polycarbonates also generally have a broad distribution of molar masses (high polydispersity, PDI). Furthermore, the technical accessibility of many of the catalyst complexes described is not optimal.

The object of the present invention is to at least partially eliminate the disadvantages addressed in the prior art. It was a particular object to provide a method for reacting epoxides with carbon dioxide in which the energy input is lower and the catalyst can be readily obtained.

This object was achieved according to the invention by a method for preparing linear and/or cyclic carbonate esters comprising the step of reacting an epoxide with carbon dioxide in the presence of a catalyst, wherein the catalyst comprises a complex of a metal M or M-A with a ligand L, the metal M being present in an oxidation state of ≧0,

A is halide, carboxylate, phenolate, sulfonate, phosphonate, alkyl, alkoxy or amido

and the ligand L has the following structure (Ia) or (Ib), wherein one or both of the OH groups shown in (Ia) and/or (Ib) can also be deprotonated:

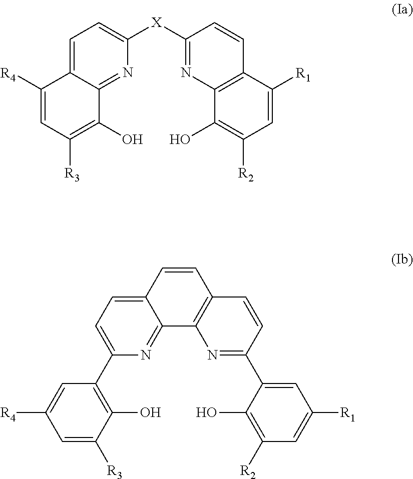

where R1, R2, R3 and R4 are each independently hydrogen, a C₁-C₂₂-alkyl residue, a C₅-C₁₂-cycloalkyl residue, a C₇-C₁₄-aralkyl or -alkylaryl residue or a C₆-C₁₄-aryl residue and X is a bridge of the formula (II) or (III):

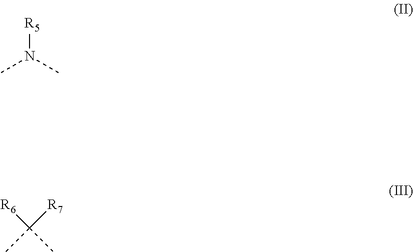

where R5 is a C₁-C₂-alkyl residue, a C₅-C₁₂-cycloalkyl residue, a C₇-C₁₄-aralkyl or -alkylaryl residue, a C₆-C₁₄-aryl residue, or an optionally alkyl-substituted pyridyl residue or pyridylmethyl residue and
R6 and R7 are each independently hydrogen, a C₁-C₂₂-alkyl residue, a C₅-C₁₂-cycloalkyl residue, a C₇-C₁₄-aralkyl or -alkylaryl residue, a C₆-C₁₄-aryl residue or a —COOR8 residue, where R8 is a C₁-C₈-alkyl residue.

The term “alkyl” in the context of this invention comprises acyclic saturated or unsaturated aliphatic hydrocarbyl residues which, in each case, may be branched or unbranched and also unsubstituted or mono- or polysubstituted. “Alkyl” is preferably selected from the group comprising methyl, ethyl, n-propyl, 2-propyl, n-butyl, isobutyl, sec-butyl, tert-butyl, n-pentyl, isopentyl, neopentyl, n-hexyl, n-heptyl, n-octyl, n-nonyl, n-decyl, ethenyl (vinyl), ethynyl, propenyl (—CH₂CH═CH₂, —CH═CH—CH₃, —C(═CH₂)—CH₃), propynyl (—CH₂—C≡CH, —C≡C—CH₃), butenyl, butynyl, pentenyl, pentynyl, hexenyl and hexynyl, heptenyl, heptynyl, octenyl, octynyl, nonenyl, nonynyl, decenyl and decynyl.

For the purposes of this invention, the term “cycloalkyl” means cyclic aliphatic (cycloaliphatic) hydrocarbons, in which the hydrocarbons may be saturated or unsaturated (but not aromatic), unsubstituted or mono- or polysubstituted. The cycloalkyl can bond to the respective high-level general structure via any desired and possible ring member of the cycloalkyl residue. The cycloalkyl residues may also be condensed with further saturated, (partially) unsaturated, (hetero)cyclic, aromatic or heteroaromatic ring systems, i.e. with cycloalkyl, heterocyclyl, aryl or heteroaryl, which may in turn be unsubstituted or mono- or polysubstituted. The cycloalkyl residues may further contain one or more bridges, for example in the case of adamantyl, bicyclo[2.2.1]heptyl or bicyclo[2.2.2]octyl. Cycloalkyl is preferably selected from the group comprising cyclopropyl, cyclobutyl, cyclopentyl, cyclohexyl, cycloheptyl, cyclooctyl, cyclononyl, cyclodecyl, adamantyl,

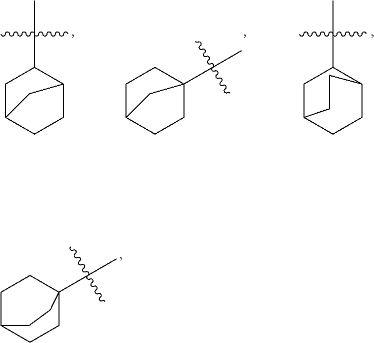 [SMILES: *C1CC2CCC1C2 ; *C12CCC(CC1)C2 ; *C1CC2CCC1CC2 ; *C12CCC(CC1)CC2]

cyclopentenyl, cyclohexenyl, cycloheptenyl and cyclooctenyl.

In the context of this invention, the term “aryl” means aromatic hydrocarbons, inter alia, phenyls and naphthyls. Each aryl residue may be present unsubstituted or in mono- or polysubstituted form, in which the aryl substituents may be the same or different and in any desired and possible position of the aryl. The aryl can bond to the high-level general structure via any desired and possible ring member of the aryl residue. The aryl residues may also be condensed with further saturated, (partially) unsaturated, (hetero)cyclic, aromatic or heteroaromatic ring systems, i.e. with cycloalkyl, heterocyclyl, aryl or heteroaryl, which may in turn be unsubstituted or mono- or polysubstituted. Examples of condensed aryl residues are benzodioxolanyl and benzodioxanyl. Aryl is preferably selected from the group comprising phenyl, 1-naphthyl and 2-naphthyl, which may in each case be unsubstituted or mono- or polysubstituted. A particularly preferred aryl is phenyl, which may be unsubstituted or mono- or polysubstituted.

In a corresponding manner, the terms “aralkyl” and “alkylaryl” derive from combinations of “alkyl” and “aryl”.

In general, for the method according to the invention, it is possible to use epoxides having 2-24 carbon atoms. The epoxides having 2-24 carbon atoms are, for example, one or more compounds selected from the group consisting of ethylene oxide, propylene oxide, 1-butene oxide, 2,3-butene oxide, 2-methyl-1,2-propene oxide (isobutene oxide), 1-pentene oxide, 2,3-pentene oxide, 2-methyl-1,2-butene oxide, 3-methyl-1,2-butene oxide, 1-hexene oxide, 2,3-hexene oxide, 3,4-hexene oxide, 2-methyl-1,2-pentene oxide, 4-methyl-1,2-pentene oxide, 2-ethyl-1,2-butene oxide, 1-heptene oxide, 1-octene oxide, L-nonene oxide, 1-decene oxide, 1-undecene oxide, 1-dodecene oxide, 4-methyl-1,2-pentene oxide, butadiene monoxide, isoprene monoxide, cyclopentene oxide, cyclohexene oxide, cycloheptene oxide, cyclooctene oxide, limonene oxide, styrene oxide, methylstyrene oxide, pinene oxide, mono- or polyepoxidized fats as mono-, di- and triglycerides, epoxidized fatty acids, C1-C24 esters of epoxidized fatty acids, epichlorohydrin, glycidol, and derivatives of glycidol, for example methyl glycidyl ether, ethyl glycidyl ether, 2-ethylhexyl glycidyl ether, allyl glycidyl ether, glycidyl methacrylate and epoxy-functional alkoxysilanes, for example 3-glycidyloxypropyltrimethoxysilane, 3-glycidyloxypropyltriethoxysilane, 3-glycidyloxypropyltripropoxysilane, 3-glycidyloxypropylmethyldimethoxysilane, 3-glycidyloxypropylethyldiethoxysilane, 3-glycidyloxypropyltriisopropoxysilane.

Various epoxides may also be used as monomers, in which both mixtures and sequential additions are possible.

The CO₂ pressure for reacting the epoxides with CO₂ is, for example, ≧1 bar to ≦80 bar, preferably ≧2 bar to ≦50 bar.

The reaction temperatures for the reaction of the epoxides with CO₂ are, for example, ≧0° C. to ≦150° C., preferably ≧10° C. to ≦130° C. Due to the comparatively low potential reaction temperatures, it is possible, better than in processes according to the prior art, to use epoxides having thermolabile functionalities such as alkoxysilanes or double bonds as comonomers, without causing undesired reactions which can lead to crosslinking.

The reaction may be carried out in batch mode, or in a semi-continuous or continuous process, in which the reactants may be dispensed in any sequence.

Mixtures of complexes with various ligands of the formula (Ia)/(Ib) and different central ions may also be used as catalyst.

If the oxidation number of the central ion is greater than +II, the excess charge may be compensated by a suitably selected anion. In this case, it is possible to use, for example, halides, carboxylates, phenolates, sulfonates, phosphonates, alkyl residues, alkoxy residues or amido compounds. In the carboxylates, aliphatic or aromatic residues thereof may be substituted by fluorine. The phenolates may be substituted by electron-withdrawing substituents such as nitro, fluorine, chlorine, ester groups or trifluoromethyl.

Non-occupied coordination sites in the catalyst complexes may be saturated by solvent molecules or additional ligands such as pyridine, acetonitrile, tetrahydrofuran, alcohols used as starter, the resulting polycarbonate, or the cocatalyst.

The reaction may be carried out in the absence or presence of solvents, for example, in ethers, in cyclic ethers such as tetrahydrofuran or dioxane, in halogenated hydrocarbons such as dichloromethane, dichloroethane, dichloroethylene, chlorobenzene or dichlorobenzene, in aromatic compounds such as benzene or toluene, in nitriles such as acetonitrile or in dipolar aprotic solvents such as dimethylformamide, N-methyl-2-pyrrolidone, triethyl phosphate or dimethyl sulfoxide.

The complexes according to the invention are technically simple to access and the ligands used according to the invention enable the simple synthesis of a catalyst library with different central ions. The reaction of epoxides and CO₂ proceeds by catalysis with the complexes according to the invention even at low temperatures.

The present invention is described below with further embodiments and aspects. They can be combined as desired, unless the opposite is clear from the context.

In one embodiment of the method according to the invention, A is OCOCH₃, OCOCF₃, OSO₂CF₃, OSO₂C6H₅CH₃, or halide.

In a further embodiment of the method according to the invention, M is selected from the group of Zn(II), Cr(II), Mn(II), Mg(II), Fe(II), Co(II), Cr(III)-A, Mn(III)-A, Fe(III)-A and/or Co(III)-A; M is preferably selected from the group of Zn(II), Cr(III)-A and/or Co(III)-A.

In a further embodiment of the method according to the invention, the ligand L is selected from the group comprising compounds of the formulae (IV) to (VII):

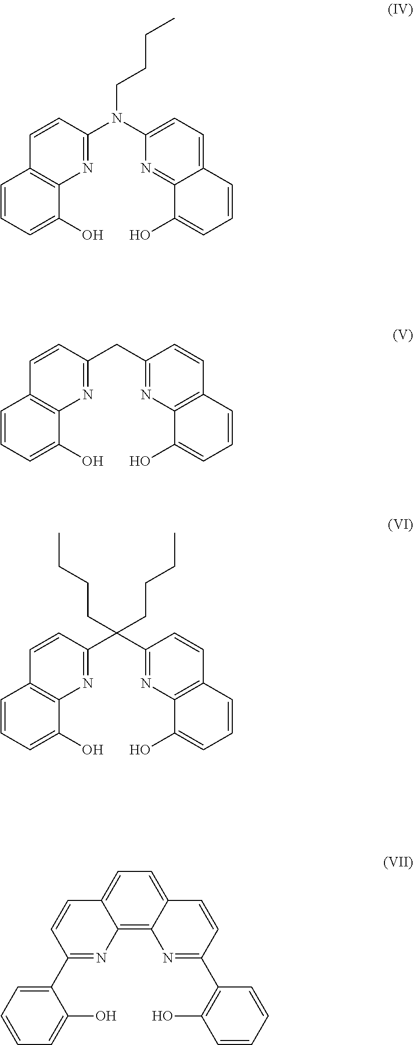 [SMILES: CCCCN(c1ccc2cccc(O)c2n1)c1ccc2cccc(O)c2n1 ; Oc1cccc2ccc(Cc3ccc4cccc(O)c4n3)nc12 ; CCCCC(CCCC)(c1ccc2cccc(O)c2n1)c1ccc2cccc(O)c2n1 ; Oc1ccccc1-c1ccc2ccc3ccc(-c4ccccc4O)nc3c2n1]

In a further embodiment of the method according to the invention, the catalyst is selected from the group comprising compounds of the formulae (VIII) to (XVIII):

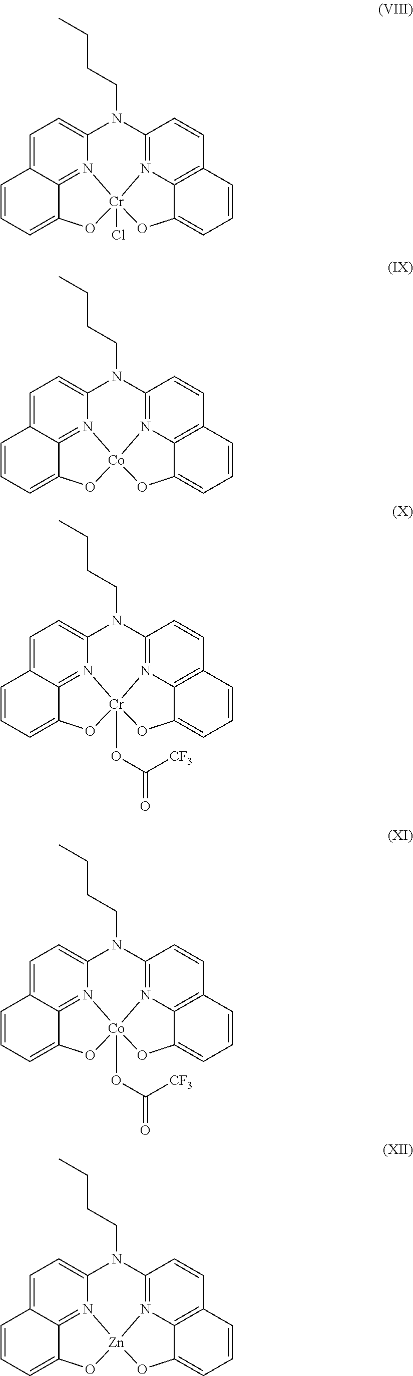

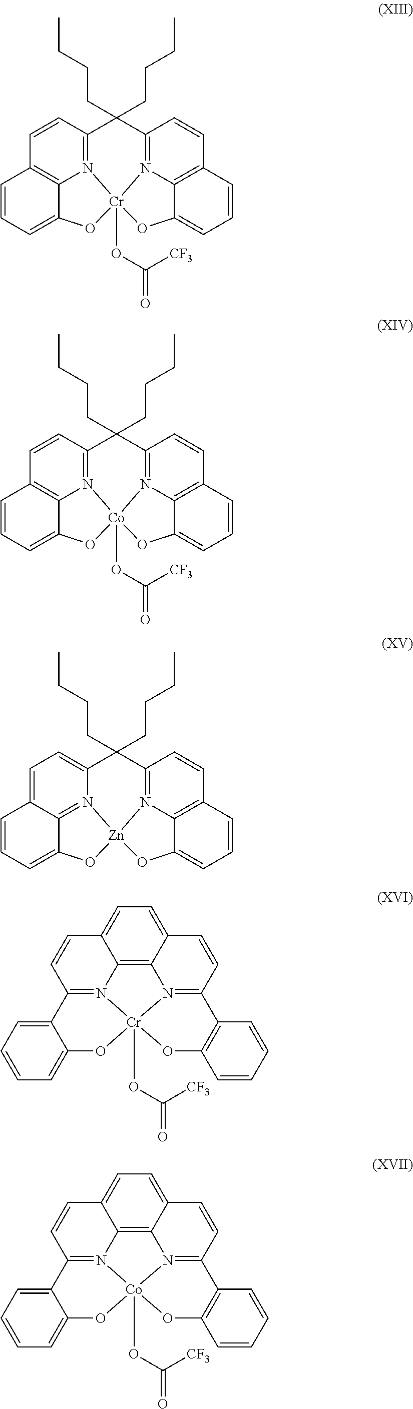

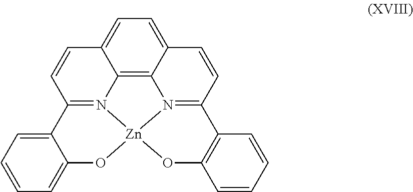

In a further embodiment of the method according to the invention, the epoxide used is ethylene oxide, propylene oxide, cyclohexene oxide or styrene oxide or any mixture comprising ethylene oxide, propylene oxide, cyclohexene oxide and styrene oxide.

In a further embodiment of the method according to the invention, the reaction is conducted at a temperature of ≧60° C. to ≦120° C. The reaction temperature is preferably ≧60° C. to ≦100° C.

In a further embodiment of the method according to the invention, the reaction mixture comprising catalyst is heated to a temperature of ≧40° C. to ≦150° C. prior to contact with epoxide. It is also possible to treat the reaction mixture comprising catalyst with a portion of the epoxide and to heat to a temperature of ≧40° C. to ≦150° C.

The catalyst may be activated under reduced pressure, CO₂ or an inert gas such as nitrogen or argon. The pressures during the activation are generally ≧0.001 bar to ≦100 bar, preferably ≦0.01 bar to ≦50 bar. It is also possible to conduct the activation in the presence of alcohols used as starters (see below) and/or epoxides, in which these may also be added in the course of the activation.

After the activation, the temperature is preferably lowered to below 80° C., before the actual reaction begins.

In an alternative method, the subsequent reaction of epoxides and CO₂ is carried out at temperatures of 0 to 150° C., preferably 10 to 100° C.

In a further embodiment of the method according to the invention, a co-catalyst from the group of ammonium salts, phosphonium salts, bis(triphenylphosphine)iminium salts, amidines, guanidines or DMAP (4-(dimethylamino)pyridine) is also used in the reaction.

Such co-catalysts may be added, for example, in amounts of ≧0.1 mol to ≦10 mol, based on the number of moles of catalyst used.

Particularly preferred co-catalysts are PPNCl (bis(triphenylphosphoranylidene)ammonium chloride), TBACl (tetrabutylammonium chloride), and/or TBABr (tetrabutylammonium bromide).

Preferred guanidines are DBU (1,8-diazabicyclo[5.4.0]undec-7-ene), DBN (1,5-diazabicyclo[4.3.0]non-5-ene) or TBD (1,5,7-triazabicyclo[4.4.0]dec-5-ene).

In a further embodiment of the method according to the invention, the reaction is conducted in the presence of an alcohol having ≧1 to ≦8 OH groups.

Such alcohols used as starters may have molar masses of ≧31 g/mol to ≦2000 g/mol. The residue may be aliphatic, aromatic, cycloaliphatic or araliphatic or comprise ether groups. Preference is given to octanediol (particularly 1,8-octanediol), decanediol, dodecanediol, dimer fatty diol, polypropylene glycols or polytetrahydrofurans. Starters are preferably charged in the activation, but they can also be added during the activation or at the beginning of the actual reaction.

A further aspect of the present invention is a polymeric carbonate ester, obtainable by reacting a cyclic epoxide with carbon dioxide in a method according to the invention, in which the number-average molecular weight Mₙ, determined by gel permeation chromatography, is in a range from ≧500 g/mol to ≦50 000 g/mol, preferably in a range from ≧500 g/mol to ≦5000 g/mol and the polydispersity index M_{w}/Mₙ is in a range from ≧1.0 to ≦1.5, preferably ≧1.1 to ≦1.4. The molecular weight may be determined against polystyrene standards using THF as eluent according to DIN 55672-1.

In this case, the cyclic epoxide is preferably cyclohexene oxide.

The present invention also relates to the use of complexes of a metal M or M-A with a ligand L as catalysts for the reaction of an epoxide with carbon dioxide, wherein the metal M is present in an oxidation state of ≧0,

A is halide, carboxylate, phenolate, sulfonate, phosphonate, alkyl, alkoxy or amido

and the ligand L has the following structure (Ia) or (Ib), wherein one or both of the OH groups shown in (Ia) and/or (Ib) can also be deprotonated:

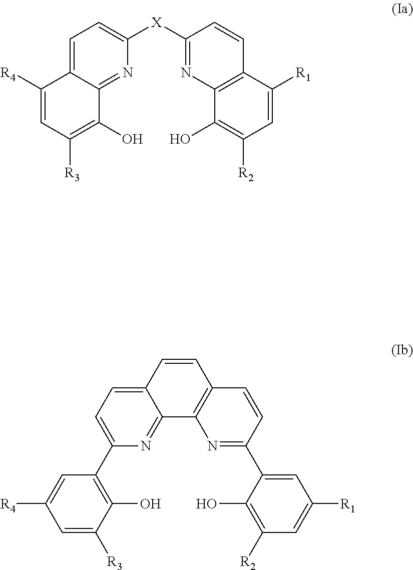

where R1, R2, R3 and R4 are each independently hydrogen, a C₁-C₂₂-alkyl residue, a C₅-C₁₂-cycloalkyl residue, a C₇-C₁₄-aralkyl or -alkylaryl residue or a C₆-C₁₄-aryl residue and X is a bridge of the formula (II) or (III):

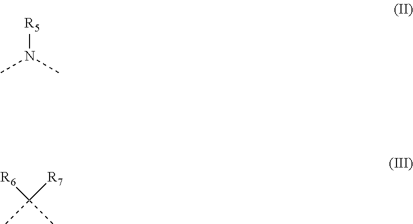

where R5 is a C₁-C₂₂-alkyl residue, a C₅-C₁₂-cycloalkyl residue, a C₇-C₁₄-aralkyl or -alkylaryl residue, a C₆-C₁₄-aryl residue, or an optionally alkyl-substituted pyridyl residue or pyridylmethyl residue and

R6 and R7 are each independently hydrogen, a C₁-C₂₂-alkyl residue, a C₅-C₁₂-cycloalkyl residue, a C₇-C₁₄-aralkyl or -alkylaryl residue, a C₆-C₁₄-aryl residue or a —COOR8 residue, where R8 is a C₁-C₈-alkyl residue.

Preferred embodiments of the ligands and catalysts, which have been elucidated with reference to the method according to the invention, are also evidently included in the use according to the invention.

A further aspect of the present invention are metal complexes according to the formula (XIX), in which the metal M is Cr(III)-A, Co(III)-A and/or Zn(II) and A is halide, carboxylate, phenolate, sulfonate, phosphonate, alkyl, alkoxy or amido:

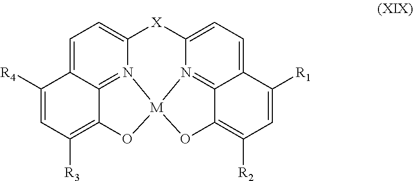

where R1, R2, R3 and R4 are each independently hydrogen, a C₁-C₂₂-alkyl residue, a C₅-C₁₂-cycloalkyl residue, a C₇-C₁₄-aralkyl or -alkylaryl residue or a C₆-C₁₄-aryl residue and X is a bridge of the formula (II) or (III):

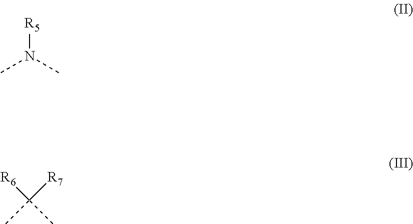

where R5 is a C₁-C₂₂-alkyl residue, a C₅-C₁₂-cycloalkyl residue, a C₇-C₁₄-aralkyl or -alkylaryl residue, a C₆-C₁₄-aryl residue, or an optionally alkyl-substituted pyridyl residue or pyridylmethyl residue and

R6 and R7 are each independently hydrogen, a C₁-C₂₂-alkyl residue, a C₅-C₁₂-cycloalkyl residue, a C₇-C₁₄-aralkyl or -alkylaryl residue, a C₆-C₁₄-aryl residue or a —COOR8 residue, where R8 is a C₁-C₈-alkyl residue;

or metal complexes of the formula according to the formula (XX), in which the metal M is Cr(III)-A, Co(III)-A and/or Zn(II) and A is halide, carboxylate, phenolate, sulfonate, phosphonate, alkyl, alkoxy or amido:

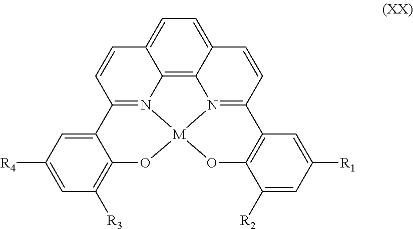

where R1, R2, R3 and R4 are each independently hydrogen, a C₁-C₂₂-alkyl residue, a C₅-C₁₂-cycloalkyl residue, a C₇-C₁₄-aralkyl or -alkylaryl residue or a C₆-C₁₄-aryl residue.

These metal complexes are preferably selected from the group comprising compounds of the formulae (XXI) to (XXVI):

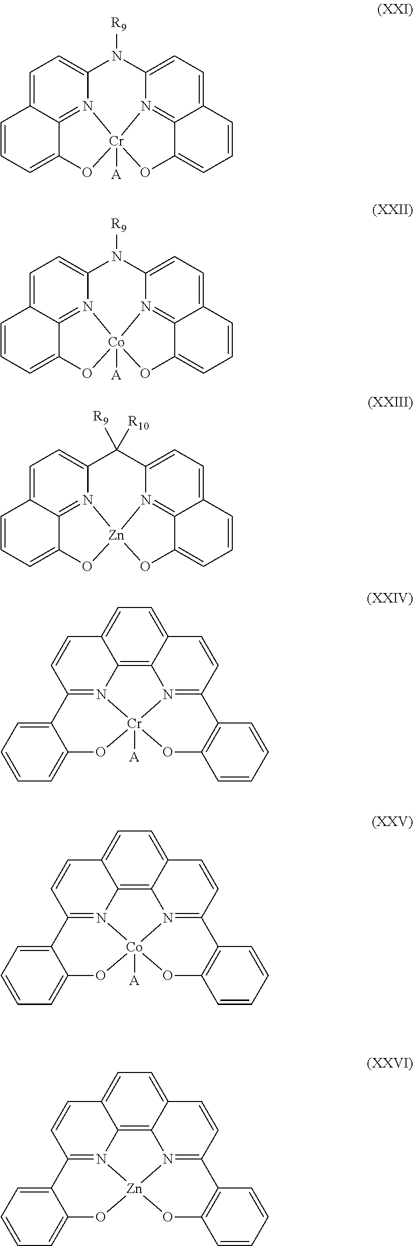

where R9 and R10 are each independently hydrogen, methyl, ethyl, propyl, butyl, pentyl or hexyl and A is OCOCH₃, OCOCF₃, OSO₂CF₃, OSO₂C₆H₅CH₃ or halide.

### EXAMPLES

The invention is illustrated in more detail by the figures and examples which follow, but without being restricted thereto.

The values for pressure refer to the absolute pressure.

The 160 mL pressure reactor used in the examples had a height (internal) of 5.08 cm and an internal diameter of 6.35 cm. The reactor was equipped with an electrical heating jacket (maximum heating power 525 watts). The counter-cooling consisted of an immersed tube of external diameter 3.17 mm which had been bent into a spiral shape and which projected into the reactor up to 5 mm above the base, and through which cooling water flowed at ca. 10° C. The water flow was switched on and off by means of a magnetic valve. In addition, the reactor was equipped with an inlet tube and a thermal sensor of diameter 1.6 mm, which both projected into the reactor up to 3 mm above the base.

The hollow shaft stirrer used in the examples was a hollow shaft stirrer in which the gas was introduced into the reaction mixture via a hollow shaft in the stirrer. The stirrer body mounted on the hollow shaft had four arms with a diameter of 50 mm and a height of 20 mm. At each end of the arm was mounted a gas outlet which had a diameter of 3 mm. The rotation of the stirrer gave rise to a reduced pressure such that the gas present above the reaction mixture (CO₂ and possibly alkylene oxide) was sucked in and introduced through the hollow shaft of the stirrer into the reaction mixture.

For recording of the epoxide concentration and the product formation during the reaction of epoxides and CO₂, a Bruker MATRIX-MF spectrometer equipped with a 3.17 mm ATR-IR fiber optic probe was used. The ATR-IR fiber optic probe (90° diamond prism with 1×2 mm basal area and 1 mm height as ATR element, 2×45° reflection of the IR beam, IR beam coupled via fiber optics) was fitted into the reactor in such a way that the diamond at the end of the 3.17 mm ATR fiber optic probe was completely immersed in the reaction mixture. IR spectra (average of 100 scans) were recorded time-resolved in the region 4000-400 cm⁻¹ with a resolution of 4 cm⁻¹. The resulting spectra were evaluated using the PEAXACT software. The propylene oxide concentration was monitored by recording the characteristic band for propylene oxide at 830 cm⁻¹. The concentration of cyclic propylene carbonate was monitored by recording the characteristic band for cyclic propylene carbonate at 1810 cm⁻¹. The cyclohexene oxide concentration was monitored by recording the characteristic band for cyclohexene oxide at 815 cm⁻¹. The concentration of cyclic cyclohexene carbonate was monitored by recording the characteristic band for cyclic cyclohexene carbonate at 1820 cm⁻¹ (cis isomer) and 1804 cm⁻¹ (trans isomer). The concentration of polycyclohexene carbonate was monitored by recording the characteristic band for polycyclohexene carbonate at 1743 cm⁻¹.

The reaction mixture was characterized by ¹H NMR spectroscopy and gel permeation chromatography.

Cyclic propylene carbonate resulted from the reaction of propylene oxide and CO₂ in the present examples. The molar proportion of converted propylene oxide (C in mol %) was determined by ¹H-NMR spectroscopy. Each sample was dissolved in deuterated chloroform and analyzed on a Bruker spectrometer (AV400, 400 MHz). The relevant resonances in the ¹H NMR spectra (relative to TMS=0 ppm), which were used for integration, are as follows:
I1: 1.45-1.49: methyl group of the cyclic carbonate, resonance area corresponds to three hydrogen atoms
I2: 2.95-2.99: methine group of free, unreacted propylene oxide, resonance area corresponds to one hydrogen atom

Taking account of the relative intensities, the values were calculated as follows:

The molar proportion of the converted propylene oxide (C in mol %) based on the sum total of the amount of propylene oxide used in the activation and the copolymerization is calculated by the formula (XXVII):

C=[(I1/3)/((I1/3)+(I2/))]×100% (XXVII)

The copolymerization of cyclohexene oxide and CO₂ resulted in polycyclohexene carbonate, comprising the polycarbonate units as shown in formula (XXVIII), in addition to the cyclic cis- and trans-cyclohexene carbonate.

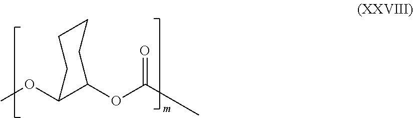

The ratio of the amount of cyclic cyclohexene carbonate to polycyclohexene carbonate (selectivity c/l) and also the molar proportion of cyclohexene oxide converted (C in mol %) were determined by ¹H-NMR spectroscopy. Each sample was dissolved in deuterated chloroform and analyzed on a Bruker spectrometer (AV400, 400 MHz). The relevant resonances in the ¹H NMR spectra (relative to TMS=0 ppm), which were used for integration, are as follows:
I3: 4.51: methine group of the polycarbonate units, resonance area corresponds to two hydrogen atoms,
I4: 3.81: methine group of the cyclic carbonate, resonance area corresponds to two hydrogen atoms,
I5: 2.98: methine group of free, unreacted cyclohexene oxide, resonance area corresponds to two hydrogen atoms.

The molar ratio of the amount of cyclic cyclohexene carbonate to carbonate units in the polycyclohexene carbonate (selectivity c/l) and also the molar proportion of the cyclohexene oxide converted (C in mol %) are reported.

Taking account of the relative intensities, the values were calculated as follows:

Molar ratio of the amount of cyclic cyclohexene carbonate to carbonate units in the polycyclohexene carbonate (selectivity c/l):

c/l=I4/I3 (XXIX)

The molar proportion of the converted cyclohexene oxide (C in mol %) based on the sum total of the amount of cyclohexene oxide used in the activation and the copolymerization is calculated by the formula:

C=[((I3/2)+(I4/2))/((I3/2)+(I4/2)+(I5/2))]×100% (XXX)

The number-average molecular weight Mₙ and the weight-average molecular weight M, of the resulting polymers were determined by means of gel permeation chromatography (GPC). The procedure of DIN 55672-1 was followed: “Gel permeation chromatography, Part 1—Tetrahydrofuran as eluent” (SECurity GPC System from PSS Polymer Service, flow rate 1.0 mL/min; columns: 2×PSS SDV linear M, 8×300 mm, 5 μm; RID detector). Here, polystyrene samples of known molar mass from PSS Polymer Service were used for the calibration.

### Example 1

### Ligand H₂(babhq)

The ligand H₂(babhq) of the formula (XXXI) was obtained from Sigma-Aldrich (CAS 82361-90-8).

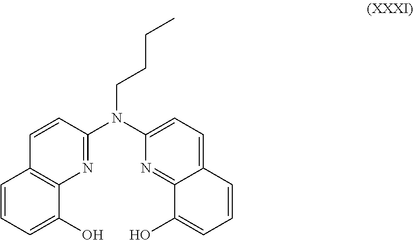 [SMILES: CCCCN(c1ccc2cccc(O)c2n1)c1ccc2cccc(O)c2n1]

### Example 2

### Synthesis of the Ligand Hz (Bpphen)

The ligand H₂(bpphen) of the formula (XXXII) was prepared in three stages starting from anisole (CAS 100-66-3). The numbers given in the formula (XXXII) refer here to the numbering of the atoms for the signal assignment in the NMR spectra. The products of the stages one to three (2-lithioanisole CAS 31600-86-9, 2,9-bis(2-methoxyphenyl)-1,10-phenanthroline CAS 127347-71-1, H₂(bpphen) CAS 192631-69-9) are known from the literature and were synthesized based on S. Routier, V. Joanny, A. Zaparucha, H. Vezin, J.-P. Catteau, J.-L. Bernier, C. Bailly, J. Chem. Soc., Perkin Trans. 2 1998, 863-868 as follows:

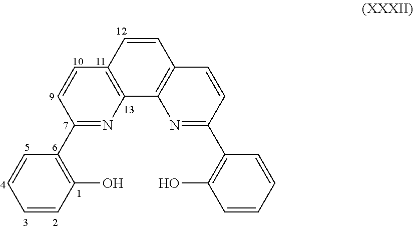 [SMILES: Oc1ccccc1-c1ccc2ccc3ccc(-c4ccccc4O)nc3c2n1]

In the first stage, 2-lithioanisole was prepared by reacting 2-bromoanisole with butyllithium according to formula (XXXIII):

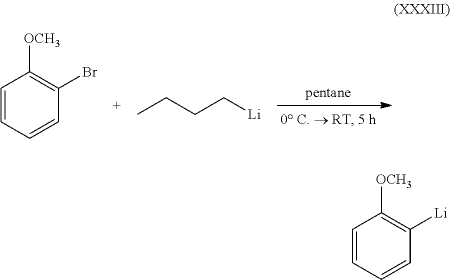

2-Bromoanisole (45.6 g, 244 mmol, 1.0 eq.) was dissolved in pentane (100 mL) and the solution was cooled to 0° C. n-Butyllithium (160 mL, 1.6M in hexane, 256 mmol, 1.1 eq.) was slowly added dropwise with vigorous stirring. A white suspension formed which was stirred at 0° C. for 30 min and subsequently for 2 h at room temperature. The white solid was separated off, washed with pentane (30 mL) and dried under fine vacuum.

Yield: 27.0 g (237 mmol, 97%), white powder.

In the second stage, 1,10-phenanthroline was reacted with the 2-lithioanisole according to formula (XXXIV) to give Me₂(bpphen) in a reaction analogous to the chichibabin reaction:

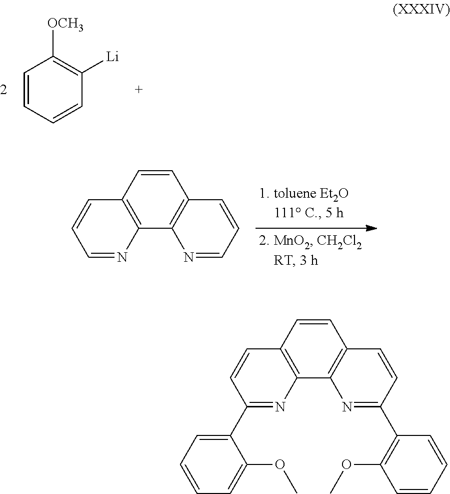

To a solution of 1,10-phenanthroline monohydrate (430 g, 21.7 mmol, 1.0 eq.) in toluene (150 mL) was added dropwise a solution of 2-lithioanisole (15.0 g, 131.5 mmol, 6.1 eq.) from stage one in diethyl ether (80 mL). In this case, the reaction mixture was first yellow, then greenish and finally deep red. The diethyl ether was firstly distilled off and then the mixture was heated under reflux for 5 h. Subsequently the reaction solution was quenched by addition of water (100 mL) at room temperature, the organic phase was separated and the aqueous phase was extracted four times with dichloromethane (at 50 mL). The combined organic phases were concentrated slightly and stirred for three hours over activated manganese dioxide (180 g, 2.1 mol, 97 eq. MERCK). The reaction mixture was then dried with sodium sulfate and filtered through Celite®. The filtrate was evaporated to dryness and the anisole by-product was condensed off at 80° C. under fine vacuum.

Yield: 7.61 g (19.4 mmol, 89%), yellow solid.

HR-ESI/MS (CH₃OH): m/z calculated for [C₂₆H₂₀N₂O₂+H]⁺: 393.1598. found: 393.1587; m/z calculated for [C₂₆H₂₀N₂O₂+Na]⁺: 415.1417. found: 415.1419.

¹H-NMR (300.1 MHz, CDCl₃): δ (ppm)=3.82 (s, 6H, OCH₃), 6.96 (d, J=8.2 Hz, 2H, H_{Ar}), 7.11 (dt, J=7.5, 0.9 Hz, 2H, H_{Ar}), 7.31-7.39 (m, 2H, H_{Ar}), 7.71 (s, 2H, H_{Ar}), 8.11-8.19 (m, 4H, H_{Ar}), 8.23 (dd, J=7.6, 1.8 Hz, 2H, H_{Ar}).

¹³C{¹H}-NMR (75.5 MHz, CDCl₃): δ (ppm)=55.8 (OCH₃), 111.6 (C_{Ar}), 121.4 (C_{Ar}), 124.8 (C_{Ar}), 125.9 (C_{Ar}), 127.4 (C_{Ar}), 129.9 (C_{Ar}), 130.2 (C_{Ar}), 132.5 (C_{Ar}), 135.2 (C_{Ar}), 146.3 (C_{Ar}), 156.3 (C_{Ar}), 157.6 (C_{Ar}).

In the third stage, the compound was deprotected using sodium ethanethiolate according to formula (XXXV).

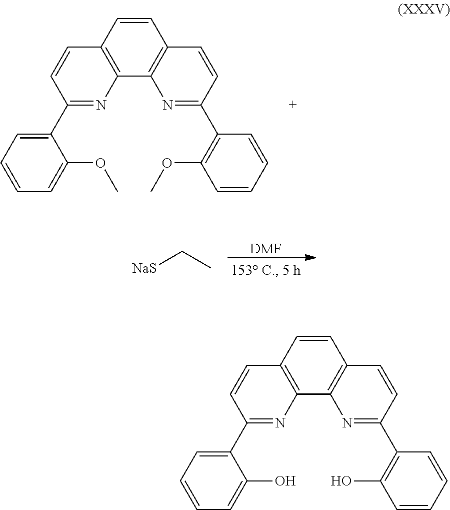

To a red solution of Me₂(bpphen) (7.61 g, 19.4 mmol, 1.0 eq.) from stage two in dimethylformamide (230 mL) was added sodium ethanethiolate (8.37 g, 99.5 mmol, 1.0 eq., prepared from ethanethiol and sodium hydride), which solution became brown. The reaction mixture was then heated under reflux for 5 h, cooled and treated with a 1:1 mixture of 10% aqueous sodium hydroxide solution and 20% hydrogen peroxide solution with ice cooling until no further gas evolution was observed. After stirring at room temperature overnight, the yellow mixture was adjusted to pH=7 with 20% sulfuric acid. In this case, a yellow solid precipitated out, which was filtered off, washed with water and recrystallized from ethanol. In order to remove impurities due to salt compounds, the resulting solid was extracted with dichloromethane and again concentrated and dried under fine vacuum.

Yield: 4.27 g (11.7 mmol, 60%), yellow needles.

HR-ESI/MS (CH₃OH): m/z calculated for [C₂₄H₁₆N₂O₂+H]⁺: 365.1285. found: 365.1282; m/z calculated for [C₂₄H₁₆N₂O₂+Na]⁺: 387.1104. found: 387.1108.

¹H-NMR (300.1 MHz, CDCl₃): δ (ppm)=6.98-7.06 (m, 2H, H4), 7.09 (dd, J=8.2, 1.0 Hz, 2H, H2), 7.41 (ddd, J=8.5, 7.3, 1.5 Hz, 2H, H3), 8.03 (s, 2H, H12), 8.25 (dd, J=8.0, 1.5 Hz, 2H, H5), 8.56 (d, J=8.8 Hz, 2H, H9), 8.65 (d, J=8.7 Hz, 2H, H10), 13.94 (s, 2H, OH).

¹³C{¹H}-NMR (75.5 MHz, CDCl₃): δ (ppm)=117.8 (C2), 119.1 (C4), 120.3 (C6), 121.3 (C9), 126.2 (C12), 127.3 (C11), 128.6 (C5), 131.9 (C3), 138.2 (C10), 141.5 (C13), 157.1 (C7), 159.1 (C1).

### Example 3

### Synthesis of the Ligand H₂(Nbhq)

The numbers refer to the numbering for the signal assignment in the NMR spectra.

The ligand H₂(nbhq) of the formula (XXXVI) was prepared in six stages starting from 8-hydroxyquinoline (CAS 148-24-3). The products of the stages one to four (1-oxy-8-hydroxyquinoline CAS 1127-45-3, dimethyl-2,2-bis(8-acetoxyquinolin-2-yl) malonate CAS 69618-69-5, H₂(mbhq) CAS 63969-39-1, [Ni(mbhq)]CAS 69595-98-8) are known from the literature and were synthesized based on Y. Yamamoto, A. Miura, A. Kawamata, M. Miura, S. Takei, Bull. Chem. Soc. Jpn. 1978, 51, 3489-3495 as follows:

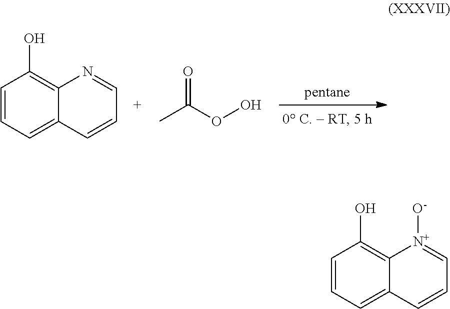

In the first stage, 8-hydroxyquinoline was oxidized with peroxyacetic acid according to formula (XXXVII):

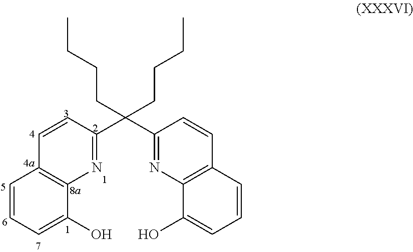 [SMILES: CCCCC(CCCC)(c1ccc2cccc(O)c2n1)c1ccc2cccc(O)c2n1]

To a solution of 8-hydroxyquinoline (50.0 g, 344 mmol) in methylene chloride (325 mL) was added dropwise at 0° C. dilute peroxyacetic acid (Fluka, ˜39% in aqueous acetic acid, 75.1 mL, 440 mol), wherein the yellow solution became deep red. After warming and stirring at room temperature for 5 h, a solution of sodium disulfite (14.0 g, 74 mmol) in water (20 mL) was added dropwise. Here the solution turned dark yellow to black. The organic phase was washed successively with 1M hydrochloric acid (ca. 300 mL), sat. sodium hydrogen carbonate solution (ca. 200 mL), sat. potassium carbonate solution (ca. 150 mL) and sat. sodium chloride solution (ca. 200 mL). The yellow-brown solution was treated with silica gel (silica gel 60 for column chromatography, 0.063-0.200 mm, 5 g) and dried with sodium sulfate. After stirring at room temperature for 15 min, the solid was separated off and the solvent was removed on a rotary evaporator. A yellow to light-brownish solid was obtained, which was dried under fine vacuum.

Yield: 42.4 g (263 mmol, 76%), yellow solid.

HR-ESI/MS (CH₃OH): m/z calculated for [C₉H₇NO₂+H]⁺: 162.0550. found: 162.0553; m/z calculated for [C₉H₇NO₂+Na]⁺: 184.0374. found: 184.0374.

¹H-NMR (300.1 MHz, CDCl₃): δ (ppm)=7.09 (dd, J=7.9, 1.1 Hz, 1H, H7), 7.24-7.29 (m, 2H, H3/H5), 7.51 (t, J=7.9 Hz, 1H, H6), 7.81 (dd, J=8.5, 0.6 Hz, 1H, H4), 8.26 (dd, J=6.1, 1.0 Hz, 1H, H2), 15.09 (s, 1H, OH).

¹³C{¹H}-NMR (75.5 MHz, CDCl₃): δ (ppm)=114.6 (C7), 116.6 (C5), 120.3 (C3), 129.4 (C4), 129.7 (C4a), 130.3 (C6), 132.1 (C8a), 134.3 (C2), 153.8 (C8).

CHN analysis: calculated for C₉H₇NO₂ (161.16 g/mol): C, 67.07; H, 4.38; N, 8.69 wt %. found: C, 67.14; H, 4.19; N, 8.63 wt %.

In the second stage, the reaction with dimethyl malonate was carried out according to formula (XXXVIII):

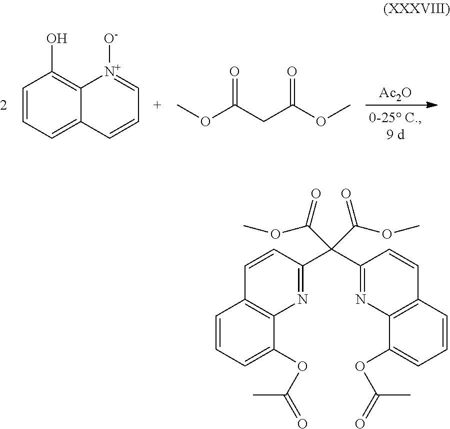

1-Oxy-8-hydroxyquinoline from stage one (13.78 g, 85.5 mmol, 1.0 eq.) and dimethyl malonate (10.8 mL, 94.2 mmol, 1.1 eq.) were suspended in acetic anhydride (50 mL) and stirred for nine days at 0-25° C. Here, the ice bath was replaced approximately every 12 h, wherein the reaction mixture warmed up in each of the intervening periods. After addition of methanol (25 mL), the yellow suspension was stirred at 0° C. for 5 h, to which distilled water (50 mL) was added and the mixture was stirred at room temperature for 20 min. The yellow precipitate was separated off, washed with 100 mL each of 50% acetic acid and distilled water and finally dried under fine vacuum. The crude product was reacted further without further purification and characterization.

Yield: 8.46 g, according to ¹H-NMR spectroscopy, 2.2:1 mixture of product to I-acetoxyquinolone, light yellow powder.

HR-ESI/MS (CH₃OH): m/z calculated for [C₂₇H₂₂N₂O₈+Na]⁺: 525.1268. found: 525.1265; m/z calculated for [C₅₄H₄₄N₄O₁₆+Na]⁺: 1027.2645. found: 1027.2664.

In stage three, the compound was deprotected according to formula (XXXIX) to give the ligand H₂(mbhq):

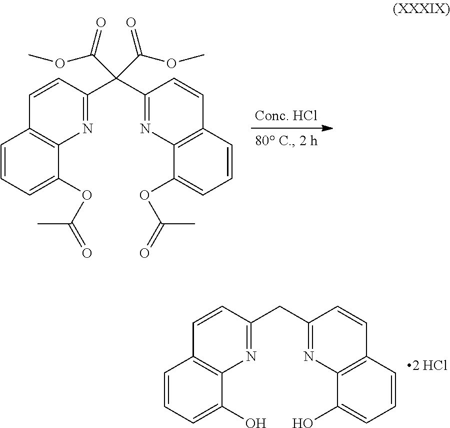 [SMILES: COC(=O)C(C(=O)OC)(c1ccc2cccc(OC(C)=O)c2n1)c1ccc2cccc(OC(C)=O)c2n1 ; Oc1cccc2ccc(Cc3ccc4cccc(O)c4n3)nc12]

The product mixture from stage two was suspended in concentrated hydrochloric acid (250 mL) and heated under reflux for 2 h. The resulting yellow precipitate was then separated off while hot and washed twice with cold hydrochloric acid (20 mL). The crude product was recrystallized from ethanol (700 mL), filtered off and washed with hydrochloric acid (1M) and distilled water and dried under fine vacuum.

Yield: 2.83 g (7.57 mmol, 18% over both stages), yellow to reddish solid.

HR-ESI/MS (CH₃OH): m/z calculated for [C₁₉H₁₄N₂O₂+H]⁺: 303.1128. found: 303.1129; m/z calculated for [C₁₉H₁₄N₂O₂+Na]⁺: 325.0947. found: 325.0944.

¹H-NMR (300.1 MHz, (CD₃)₂SO): δ (ppm)=5.27 (s, 2H, CH₂), 7.49-7.55 (m, 2H, H7), 7.58-7.70 (m, 4H, H5/H6), 7.97 (d, J=8.6 Hz, 2H, H3), 8.86 (d, J=8.6 Hz, 2H, H4). The alcohol protons were not resolved.

¹³C{¹H}-NMR (75.5 MHz, (CD₃)₂SO): δ (ppm)=39.9 (CH₂), 115.0 (C6), 117.7 (C5), 123.5 (C3), 128.3 (C4a), 129.9 (C7), 131.7 (C8a), 143.4 (C4), 149.7 (C8), 154.7 (C2).

CHN analysis: calculated for C₁₉H₁₄N₂O₂.2HCl (374.06 g/mol) C, 60.81; H, 4.30; N, 7.47 wt %. found: C, 60.02; H, 4.62; N, 7.31 wt %.

In the fourth stage, the alcohol functions of H₂(mbhq) were protected by complexing with Ni²⁺ according to formula (XXXX).

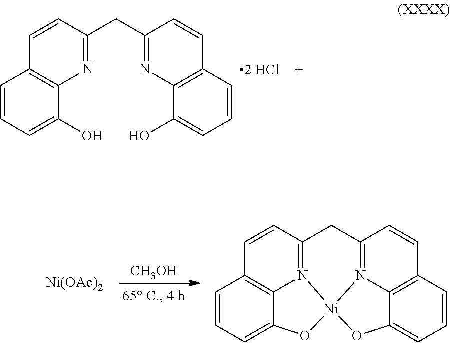

Methanol (20 mL) was charged with H₂(mbhq) from stage three (705 mg, 1.88 mmol, 1.0 eq.) and treated with nickel acetate tetrahydrate (580 mg, 2.33 mmol, 1.2 eq.). The reaction mixture was then heated under reflux for four hours, resulting in the formation of an orange-brown solid. Said solid was separated off, washed with methanol (5 mL) and dried at 50° C. under fine vacuum.

Yield: 670 mg (1.70 mmol, 90%), orange-brown solid.

HR-ESI/MS (CH₃OH): m/z calculated for [C₁₉H₁₂N₂NiO₂+Na]⁺: 381.0144. found: 381.0147.

¹H-NMR/¹³C-NMR: Not measured due to poor substance solubility.

CHN analysis: calculated for C₁₉H₁₂N₂NiO₂.2H₂O (394.05 g/mol): C, 57.77; H, 4.08; N, 7.09 wt %. found: C, 57.54; H, 3.36; N, 7.09 wt %.

In stage five, a bisalkylation of [Ni(mbhq)] with butyl iodide in the presence of sodium hydroxide was carried out according to formula (XXXXI):

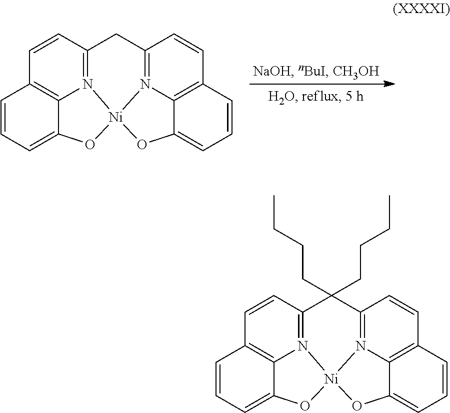

Butyl iodide (9.0 mL, 76.0 mmol, 10.0 eq.) was added to a suspension of [Ni(mbhq)] from stage four (2.70 g, 7.52 mmol, 1.0 eq.) in 100 mL of methanol at 50° C. Subsequently a solution of sodium hydroxide (0.75 g, 18.8 mmol, 2.6 eq.) in 40 mL of degassed water was added. The reaction solution was heated under reflux for five hours and then left to stand at room temperature for 12 h. Thereafter, the precipitated solid was filtered off and the solution was added to water (350 mL). The precipitate formed was likewise separated off, combined with the first, washed with hot water and dried under fine vacuum.

Yield: 3.52 g (7.47 mmol, 99%), brown solid.

HR-ESI/MS (CH₃OH): m/z calculated for [C₂₇H₂₈N₂NiO₂+Na]⁺: 493.1396. found: 493.1396; m/z calculated for [C₂₇H₂₇N₂NiO₂]⁻: 469.1431. found: 469.1438.

¹H-NMR (300.1 MHz, (CD₃)₂SO): δ (ppm)=0.57 (m, 4H, CH₂Et), 0.63 (t, J=7.3 Hz, 6H, CH₃), 1.03 (td, J=13.9, 7.0 Hz, 4H, CH₂CH₃), 2.44 (m, 4H, CH₂Pr), 6.78 (d, J=7.6 Hz, 2H, H5), 7.06 (d, J=7.7 Hz, 2H, H7), 7.42 (t, J=7.8 Hz, 2H, H6), 8.16 (d, J=8.7 Hz, 2H, H3), 8.55 (d, J=8.8 Hz, 2H, H4).

¹³C{¹H}-NMR (75.5 MHz, (CD₃)₂SO): δ (ppm)=13.3 (CH₂Et), 21.7 (CH₃), 26.3 (CH₂CH₃), 43.2 (CH₂Pr), 60.7 (CBu₂), 110.4 (C7), 113.4 (C5), 123.0 (C3), 127.9 (C4a), 130.5 (C6), 139.2 (C4), 141.8 (C8a), 158.5 (C2), 164.7 (C8).

In stage six, the butylated nickel complex was deprotected according to formula (XXXXII) to give the target ligand H₂(nbhq):

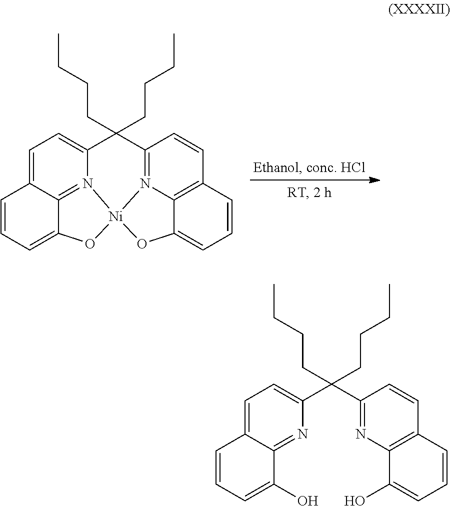

The nickel complex [Ni(nbhq)] from stage five (3.32 g, 7.05 mmol, 1.0 eq.) was suspended in ethanol (70 mL) and treated with concentrated hydrochloric acid (9 mL, 108.72 mmol, 15.4 eq.). This mixture was stirred at room temperature for two hours. The resulting precipitate was separated off and the filtrate was added to water (150 mL). After stirring at room temperature for twelve hours, the additional precipitate was separated off, washed with water and dried under fine vacuum.

Yield: 2.40 g (5.78 mmol, 82%), yellow powder.

¹H-NMR (300.1 MHz, (CD₃)₂SO): δ (ppm)=0.73 (t, J=7.3 Hz, 6H, CH₃), 0.82-0.92 (m, 4H, CH₂Et), 1.16-1.28 (m, 4H, CH₂CH₃), 2.59-2.64 (m, 4H, CH₂Pr), 7.26 (dd, J=7.4, 1.1 Hz, 2H, H7), 7.44-7.54 (m, 4H, H5/H6), 7.69 (d, J=8.6 Hz, 2H, H3), 8.45 (d, J=8.6 Hz, 2H, H4). The alcohol protons were not resolved.

¹³C{¹H}-NMR (75.5 MHz, (CD₃)₂SO): δ (ppm)=13.7 (CH₃), 22.4 (CH₂CH₃), 26.1 (CH₂Et), 54.3 (CH₂Pr), 55.9 (CBu₂), 112.4 (C6), 117.5 (C5), 121.3 (C3), 127.1 (C4a), 127.9 (C7), 134.6 (C8a), 138.7 (C4), 151.9 (C8), 162.7 (C2).

### Example 1-1

### Synthesis of the Chromium Complex [Cr(Babhq)OC(O)CF₃]

The chromium complex [Cr(babhq)OC(O)CF₃] was synthesized in two stages. In the first stage, the complex [Cr(babhq)Cl] was prepared according to formula (XXXXIII).

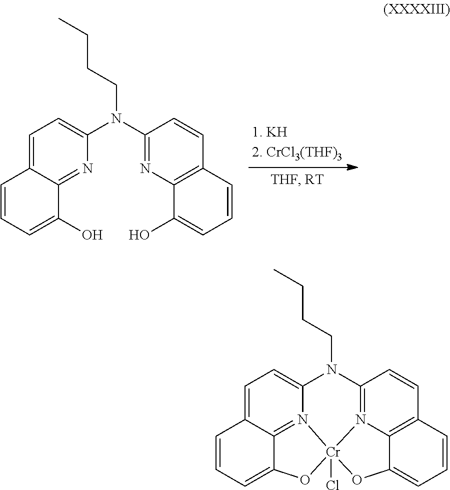

H₂(babhq) from example 1 (513 mg, 1.43 mmol, 1.0 eq.) and potassium hydride (118 mg, 2.86 mmol, 2.0 eq.) were suspended in dry THF (20 mL). This mixture was stirred until evolution of gas ceased. A suspension of purple [CrCl₃(thf)₃] (537 mg, 1.43 mmol, 1.0 eq.) in dry THF (20 mL) was added, causing the reaction mixture to turn brown. After stirring for 12 hours at room temperature, water (100 mL) was added. The precipitate formed was filtered off, washed with water, and dried at 50° C. under fine vacuum.

Yield: 545 mg (1.23 mmol, 86%), yellow solid.

HR-ESI/MS (CH₃OH): m/z calculated for [C₂₂H₁₉CrN₃O₂]⁺: 409.0878. found: 409.0879.

CHN analysis: calculated for C₂₂H₁₉CrN₃O₂.2H₂O (480.88 g/mol): C, 54.95; H, 4.82; N, 9.74 wt %. found: C, 54.84; H, 4.88; N, 9.65 wt %.

Single-crystal X-ray analysis: Crystals of the composition [Cr(babhq)Cl(dmf)].DMF were obtained by recrystallizing the compound from a saturated DMF solution by slowly cooling from 100° C. to room temperature. A single-crystal X-ray analysis of a 0.25×0.21×0.06 mm³ crystal showed that the compound crystallizes in space group P 2₁/n (monoclinic) with a=9.8802(4) Å, b=14.1274(4) Å, c=19.4439(9) Å, α=90°, β=94.197(3)°, γ=90° and 4 molecules per unit cell.

In the second stage, the complex [Cr(babhq)Cl] was reacted with silver trifluoroacetate according to formula (XXXXIV).

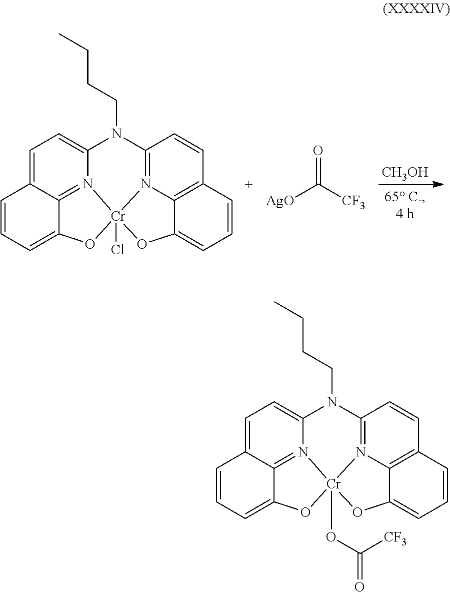

[Cr(babhq)Cl] from stage one (545 mg, 1.23 mmol, 1.0 eq.) was suspended in ethanol (100 mL). Silver trifluoroacetate (272 mg, 1.23 mmol, 1.0 eq.) was added with stirring. The mixture was boiled under reflux for 5 h. After cooling to room temperature, the solid constituents were separated by centrifugation. They were extracted repeatedly with hot ethanol. The combined solutions were evaporated to dryness and dried under fine vacuum at 50° C.

Yield: 594 mg (1.14 mmol, 92%), brown solid.

HR-ESI/MS (CH₃CN): m/z calculated for [C₂₄H₁₉CrF₃N₃O₄]⁺: 522.0727. found: 522.0740.

CHN analysis: C₂₄H₁₉CrF₃N₃O₄.EtOH (568.12 g/mol) calculated: C, 54.93; H, 4.43; N, 7.39 wt %. found: C, 54.77; H, 4.46; N, 7.43 wt %.

Single crystal X-ray analysis: Crystals of the composition [Cr(babhq)OC(O)CF₃(dmf)] were obtained as orange prisms by recrystallization of the compound from DMF/ether at room temperature. A single-crystal X-ray analysis of a 0.15×0.09×0.03 mm³ crystal showed that the compound crystallizes in space group P 2₁/c (monoclinic) with a=12.2189(9) Å, b=13.6109(9) Å, c=15.9826(11) Å, α=90°, β=102.501(5)°, γ=90° and 4 molecules per unit cell.

### Example 1-2

### Synthesis of the Cobalt Complex [Co(Babhq)OC(O)CF₃]

The cobalt complex [Co(babhq)OC(O)CF₃] was synthesized in two stages. In the first stage, the complex [Co(babhq)] was prepared according to formula (XXXXV). The cobalt(II) complex is known from Wershofen, Stefan; Klein, Stephan; Jacob, Andreas; Sundermeyer, Joerg; Mei, Fuming Ger. Offen. (2009), DE 102008006881 A1 20090806 and Jacob, Andreas; Wershofen, Stefan; Klein, Stephan; Sundermeyer, Joerg; Mei, Fuming, PCT Int. Appl. (2009), WO 2009095164 A1 20090806.

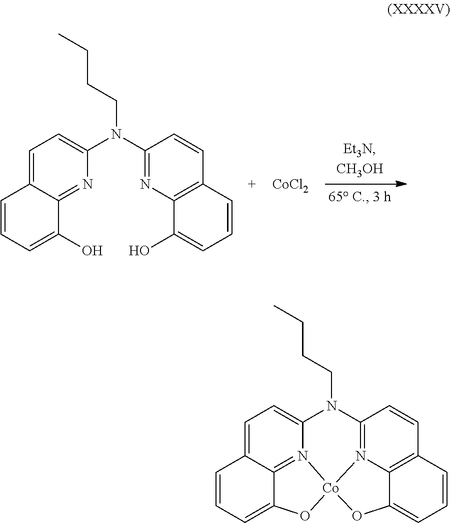

Methanol (45 mL) was charged with the ligand H₂(babhq) from example 1 (1.000 mg, 2.78 mmol, 1.0 eq.) and the mixture heated to 50° C. A solution of cobalt chloride hexahydrate (674 mg, 2.83 mmol, 1.0 eq.) in degassed water (5 mL) and triethylamine (850 L, 6.13 mmol, 2.2 eq.) was then added. There was an immediate color change to red-orange. The mixture was heated under reflux for three hours. The precipitated red solid was filtered off, washed with ether (20 mL) and dried under fine vacuum.

Yield: 870 mg (2.09 mmol, 75%), red-brown solid.

HR-ESI/MS (CH₃OH): m/z calculated for [C₂₂H₁₉CoN₃O₂]⁺: 416.0804. found: 416.0804.

CHN analysis: C₂₂H₁₉CoN₃O₂ (416.34 g/mol) calculated: C, 63.47; H, 4.60; N, 10.09 wt %. found: C, 62.94; H, 4.49; N, 9.94 wt %.

In the second stage, the Co(II) complex was oxidized with di(trifluoroacetoxy)iodosobenzene according to formula (XXXXVI).

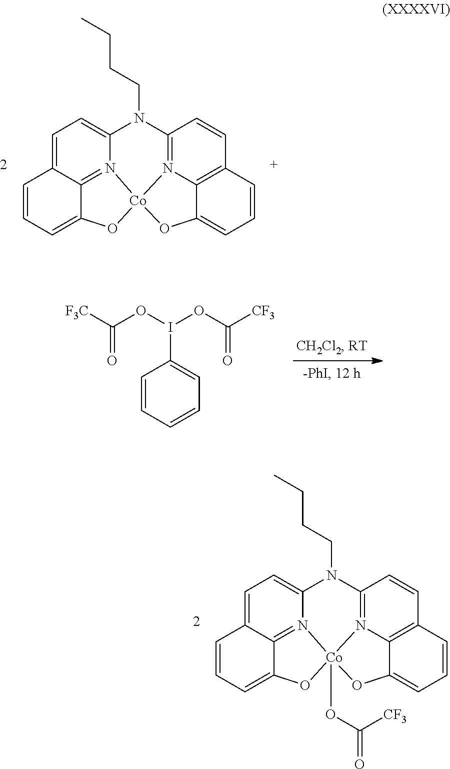

[Co(babhq)] from stage one (722 mg, 1.73 mmol, 1.9 eq.) was suspended in methylene chloride (40 mL) and treated with di(trifluoroacetoxy)iodosobenzene (392 mg, 0.91 mmol, 1.0 eq.) immediately causing the mixture to darken. This mixture was stirred overnight at room temperature. After addition of diethyl ether (10 mL), the precipitated solid was separated off, washed with diethyl ether (10 mL) and dried under fine vacuum.

Yield: 750 mg (1.42 mmol, 81%), grey-brown solid.

HR-ESI/MS (CH₃OH): m/z calculated for [C₂₄H₁₉CoF₃N₃O₄+Na]⁺: 552.0552. found: 552.0559. ¹H-NMR (300.1 MHz, (CD₃)₂SO): δ (ppm)=0.97 (t, J=7.3 Hz, 3H, CH₃), 1.50 (td, J=14.9, 7.4, 2H, CH₂CH₃), 1.86-2.07 (m, 2H, CH₂Et), 4.52-4.80 (m, 2H, NCH₂), 7.27 (dd, J=7.2, 1.7 Hz, 2H, H5), 7.45-7.66 (m, 4H, H6/H7), 7.92 (d, J=9.4 Hz, 2H, H3), 8.63 (d, J=9.4 Hz, 2H, H4).

¹³C{¹H}-NMR (62.9 MHz, (CD₃)₂SO): δ (ppm)=13.2 (CH₃), 18.4 (CH₂CH₃), 29.3 (CH₂Et), 34.0 (CF₃) 49.9 (NCH₂), 111.8 (C5), 114.2 (C3), 115.8 (C7), 125.2 (C4a), 127.9 (C6), 140.2 (C4), 145.5 (C8a), 149.4 (C2), 166.6 (C5). The carbon atom of the CF₃ group was not resolved.

CHN analysis: C₂₅H₂₀CoF₃N₂O₄.¼ CH₂Cl₂ (549.90 g/mol) calculated: C, 52.90; H, 3.57; N, 7.63 wt %. found: C, 52.74; H, 3.54; N, 7.66 wt %.

### Example 1-3

### Synthesis of the Zinc Complex [Zn(Babhq)]

The complex [Zn(babhq)] (CAS 252565-55-2) is known from Heuer, Helmut-Werner, Wehrmann, Rolf; Elschner Andreas; (Bayer AG), DE 19981025737 A1 (1999) and was prepared according to formula (XXXXVII).

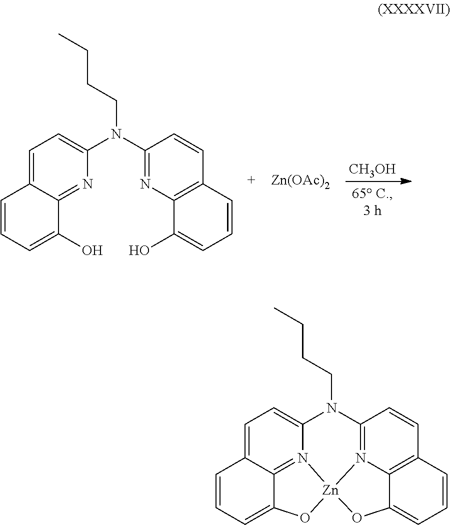

The ligand H₂(babhq) (715 mg, 2.0 mmol, 1.0 eq.) from example 1 was dissolved in 60 mL of methanol and treated with zinc acetate dihydrate (437 mg, 2.0 mmol, 1.0 eq.). The mixture was heated resulting in precipitation of a yellowish solid. After stirring for 3 h at boiling point, the mixture was cooled and the precipitated solid was separated off. This was washed with 15 mL of ether and dried under fine vacuum.

Yield: 615 mg (1.5 mmol, 73%), light-yellow powder.

HR-ESI/MS (CH₃OH): m/z calculated for [C₂₂H₂₀N₃O₂Zn]⁺: 422.0842. found: 422.0832.

¹H-NMR (300.1 MHz, (CD₃)₂SO): δ (ppm)=0.79 (t, J=7.3 Hz, 3H, CH₃), 1.31 (td, J=14.5, 7.4 Hz, 2H, CH₂CH₃), 1.61-1.74 (m, 2H, CH2Et), 4.38 (t, J=6.7 Hz, 2H, NCH2), 6.79 (dd, J=7.8, 0.9 Hz, 2H, H7), 6.88 (dd, J=7.8, 0.9 Hz, 2H, H5), 7.28 (t, J=7.9 Hz, 2H, H6), 7.66 (d, J=9.1 Hz, 2H, H3), 8.36 (d, J=9.1 Hz, 2H, H4).

¹³C{1H}-NMR (75.5 MHz, (CD₃)₂SO): δ (ppm)=13.3 (CH₃), 18.7 (CH₂CH₃), 30.2 (CH₂Et), 49.7 (NCH₂), 108.6 (C5), 112.4 (C7), 114.6 (C3), 125.1 (C4a), 127.9 (C6), 136.9 (C8a), 140.0 (C4), 151.8 (C2), 161.3 (C8).

CHN analysis: C₂₂H₁₉N₃O₂Zn.½ H₂O (431.82 g/mol) calculated: C, 61.19; H, 4.67; N, 9.73 wt %. found: C, 61.14; H, 4.61; N, 9.69 wt %.

Single crystal X-ray analysis: Crystals of the composition [Zn(babhq)CH₃OH].2CH₃OH were obtained as light yellow platelets by recrystallization of the compound from methanol/ether at room temperature. A single-crystal X-ray analysis of a 0.40×0.14×0.08 mm³ crystal showed that the compound crystallizes in space group P 2₁/n (monoclinic) with a=13.5366(8) Å, b=9.3828(14) Å, c=19.4198(14) Å, α=90°, β=93.974(5)°, γ=90° and 4 molecules per unit cell.

### Example 2-1

### Synthesis of the Chromium Complex [Cr(Bpphen)OC(O)CF₃]

The chromium complex [Cr(bpphen)OC(O)CF₃] was synthesized in two stages. In the first stage, the complex [Cr(bpphen)Cl] was prepared according to formula (XXXXVIII).

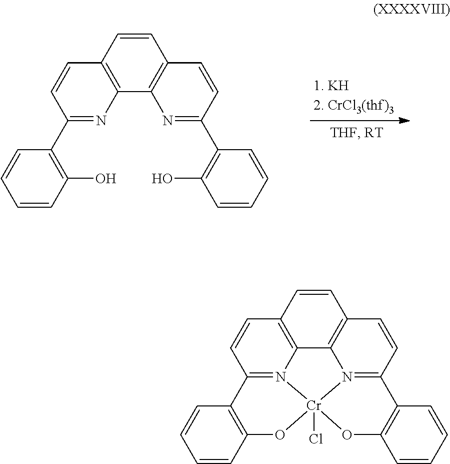

H₂(bpphen) from example 2 (500 mg, 1.37 mmol, 1.0 eq.) and potassium hydride (110 mg, 2.74 mmol, 2.0 eq.) were suspended in dry THF (20 mL). This mixture was stirred until evolution of gas ceased. A suspension of purple [CrCl₃(thf)₃] (515 mg, 1.37 mmol, 1.0 eq.) in dry THF (20 mL) was added, causing the reaction mixture to turn brown. After stirring for 12 hours at room temperature, water (100 mL) was added. The precipitate formed was filtered off, washed with water, and dried at 50° C. under fine vacuum.

Yield: 540 mg (1.20 mmol, 88%), brown solid.

HR-ESI/MS (CH₃OH): m/z calculated for [C₂₄H₁₄CrN₂O₂]⁺: 414.0455. found: 414.0452.

CHN analysis: calculated for C₂₄H₁₄CrN₂O₂.3H₂O (503.88 g/mol): C, 57.21; H, 4.00; N, 5.56 wt %. found: C, 57.47; H, 3.79; N, 5.11 wt %.

In the second stage, the complex [Cr(bpphen)Cl] was reacted with silver trifluoroacetate according to formula (IL).

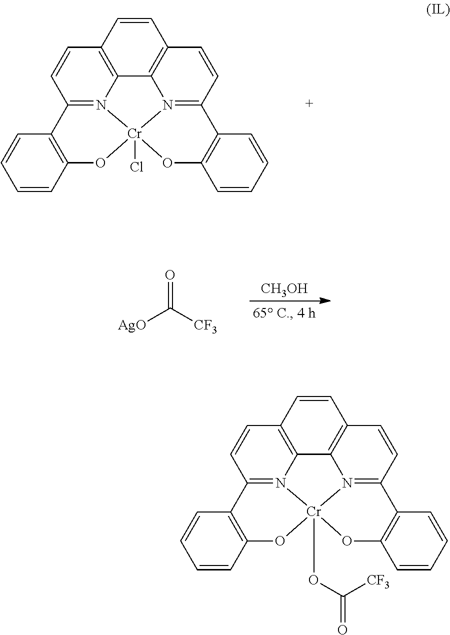

[Cr(bpphen)Cl] from stage one (128 mg, 0.29 mmol, 1.0 eq.) was suspended in ethanol (15 mL). Silver trifluoroacetate (63 mg, 0.29 mmol, 1.0 eq.) was added with stirring. The mixture was boiled under reflux for 4 h. After cooling to room temperature, the solid constituents were separated by centrifugation. They were extracted repeatedly with hot ethanol. The combined solutions were evaporated to dryness and dried under fine vacuum at 50° C.

Yield: 118 mg (0.22 mmol, 79%), brown solid.

HR-ESI/MS (CH₃CN): m/z calculated for [C₂₆H₁₄CrF₃N₂O₄+Na]⁺: 550.0204. found: 550.0203.

CHN analysis: C₂₆H₁₄CrF₃N₂O₄ (527.39 g/mol) calculated: C, 59.21; H, 2.98; N, 5.31 wt %. found: C, 49.67; H, 2.78; N, 5.18 wt %.

Single crystal X-ray analysis: Crystals of the composition [Cr(bpphen)OC(O)CF₃)]₂ were obtained as red prisms by recrystallization of the compound from DMF/ether at room temperature. A single-crystal X-ray analysis of a 0.30×0.18×0.15 mm³ crystal showed that the compound crystallizes in space group P 2₁/a (monoclinic) with a=11.2256(4) Å, b=24.1915(6) Å, c=11.4141(4) Å, α=90°, β=106.099(3)°, γ=90° and 4 molecules per unit cell.

### Example 2-2

### Synthesis of the Cobalt Complex [Co(Bpphen)OC(O)CF₃]

The cobalt complex [Co(bpphen)OC(O)CF₃] was synthesized in two stages. In the first stage, the complex [Co(bpphen)] was prepared according to formula (L). The cobalt(II) complex (CAS 208171-82-8) is known from Wershofen, Stefan; Klein, Stephan; Jacob, Andreas; Sundermeyer, Joerg; Mei, Fuming Ger. Offen. (2009), DE 102008006881 A1 20090806 and Jacob, Andreas; Wershofen, Stefan; Klein, Stephan; Sundermeyer, Joerg, Mei, Fuming, PCT Int. Appl. (2009), WO 2009095164 A1 20090806 and Orejon, Aranzazu; Castellanos, Aida; Salagre, Pilar, Castillon, Sergio; Claver, Carmen, Can. J. Chem. 2005, 83, 764-768 and also Routier, Sylvain; Joanny, Valerie; Zaparucha, Anne; Vezin, Herve; Catteau, Jean-Pierre; Bernier, Jean-Luc; Bailly, Christian, J Chem. Soc., Perkin Trans. 2 1998, 863-868.

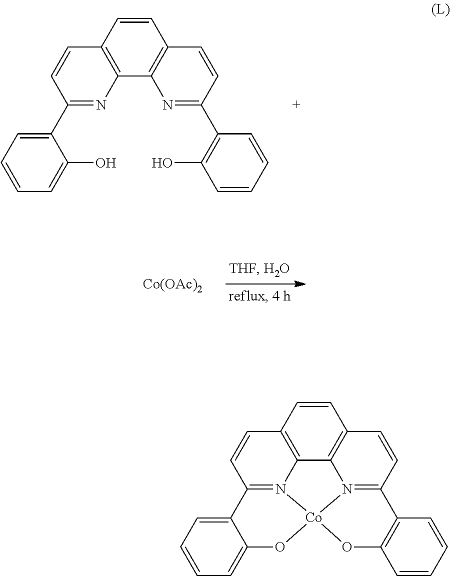

Tetrahydrofuran (40 mL) was charged with the ligand H₂(bpphen) from example 2 (984 mg, 2.70 mmol, 1.0 eq.) and the mixture heated to 50° C. Subsequently a solution of cobalt(II) acetate tetrahydrate (673 mg, 2.70 mmol, 1.0 eq.) in degassed water (10 mL) was added. There was an immediate color change to red. The mixture was heated under reflux for four hours. The precipitated red solid was filtered off, washed with ether (20 mL) and dried under fine vacuum.

Yield: 946 mg (2.25 mmol, 83%), red solid.

HR-ESI/MS (CH₃OH): m/z calculated for [C₂₄H₁₄CoN₂O₂]⁺: 421.0382. found: 421.0385.

CHN analysis: C₂₄H₁₄CoN₂O₂ (421.31 g/mol) calculated: C, 68.42; H, 3.35; N, 6.65 wt %. found: C, 67.86; H, 3.37; N, 6.55 wt %.

In the second stage, the Co(II) complex was oxidized with di(trifluoroacetoxy)iodosobenzene according to formula (LI).

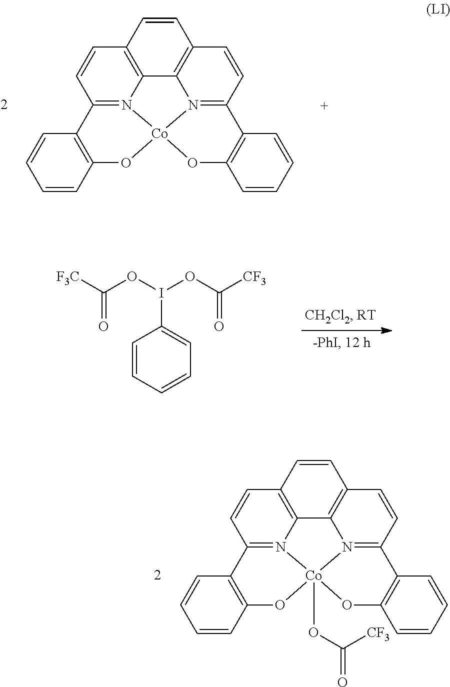

[Co(bpphen)] from stage one (506 mg, 1.20 mmol, 1.9 eq.) was suspended in tetrahydrofuran (20 mL) and treated with di(trifluoroacetoxy)iodosobenzene (268 mg, 0.62 mmol, 1.0 eq.), immediately causing the mixture to darken. This mixture was stirred overnight at room temperature. After addition of diethyl ether (10 mL), the precipitated solid was separated off, washed with diethyl ether (10 mL) and dried under fine vacuum.

Yield: 550 mg (1.03 mmol, 86%), brown solid.

HR-ESI/MS (CH₃OH): m/z calculated for [C₂₆H₁₄CoF₃N₂O₄+Na]⁺: 557.0130. found: 557.0147.

¹H-NMR (300.1 MHz, (CD₃)₂SO): δ (ppm)=6.88 (t, J=7.3 Hz, 2H, H4), 7.47 (t, J=7.5 Hz, 2H, H3), 7.87 (d, J=7.9 Hz, 2H, H2), 8.37 (s, 2H, H12), 8.41 (m, J=7.4 Hz, 2H, H5), 8.93 (d, J=8.9 Hz, 2H, H9), 9.01 (d, J=8.9 Hz, 2H, H10).

¹³C{¹H}-NMR (62.9 MHz, (CD₃)₂SO): δ (ppm)=116.3 (C4), 117.2 (C6), 121.9 (C9), 124.6 (C2), 125.9 (C12), 127.5 (C11), 129.2 (C5), 132.9 (C3), 138.6 (C10), 147.7 (C13), 155.3 (C7), 165.1 (CI). The carbon atom of the CF₃ group was not resolved.

CHN analysis: C₂₅H₁₄CoF₃N₂O₄ (534.33 g/mol) calculated: C, 58.44; H, 2.64; N, 5.24 wt %. found: C, 58.01; H, 2.71; N, 5.14 wt %.

### Example 2-3

### Synthesis of the Zinc Complex [Zn(Bpphen)]

The ligand H₂(bpphen) (700 mg, 1.9 mmol, 1.0 eq.) from example 2 was dissolved in 40 mL of methanol and treated with zinc acetate dihydrate (422 mg, 1.9 mmol, 1.0 eq.) according to formula (LII). The mixture was heated resulting in precipitation of a yellowish solid. After stirring for 3 h at boiling point, the mixture was cooled and the precipitated solid was separated off. This was washed with 10 mL of ether and dried under fine vacuum.

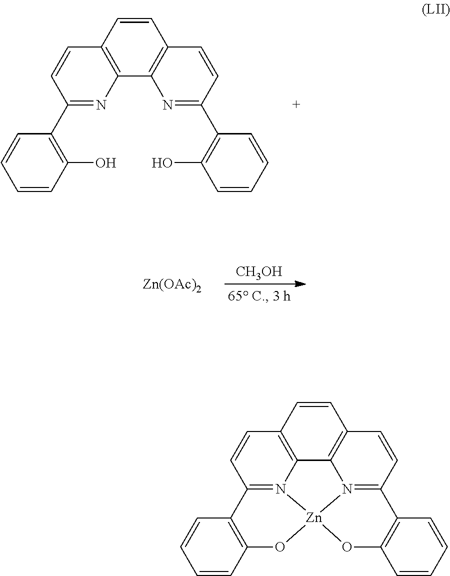

Yield: 661 mg (1.6 mmol, 80%), yellow powder.

HR-APCI/MS (CH₃OH): m/z calculated for [C₂₄H₁₄N₂O₂Zn+H]⁺: 427.0420. found: 427.0420.

¹H-NMR (300.1 MHz, (CD₃)₂SO): δ (ppm)=6.57 (ddd, J=8.1, 6.9, 1.3 Hz, 2H, H4), 6.83 (dd, J 8.4, 1.2 Hz, 2H, H2), 7.22 (ddd, J=8.5, 6.9, 1.7 Hz, 2H, H3), 7.93 (dd, J=8.2, 1.7 Hz, 2H, H5), 8.03 (s, 2H, H12), 8.42 (d, J=9.1 Hz, 2H, H9), 8.65 (d, J=8.9 Hz, 2H, H10).

¹³C{¹H}-NMR (75.5 MHz, (CD₃)₂SO): 1133 (C4), 119.7 (C6), 122.6 (C9), 124.3 (C2), 124.8 (C12), 125.4 (C11), 129.8 (C5), 132.3 (C3), 138.2 (C13), 138.5 (C10), 158.6 (C7), 170.2 (C1).

CHN analysis: C₂₄H₁₄N₂O₂Zn (427.79 g/mol) calculated: C, 67.38; H, 3.30; N, 6.55 wt %. found: C, 66.82; H, 3.40; N, 6.47 wt %.

### Example 3-1

### Synthesis of the Zinc Complex [Zn(Nbhq)]

The ligand H₂(nbhq) (630 mg, 1.52 mmol, 1.0 eq.) from example 3 was dissolved in methanol (60 mL) and zinc acetate dihydrate (334 mg, 1.52 mmol, 1.0 eq.) was added according to formula (LIII). This reaction mixture was boiled under reflux for two hours. After cooling to room temperature, a precipitate was separated off by filtration, washed with ether (15 mL) and dried under fine vacuum.

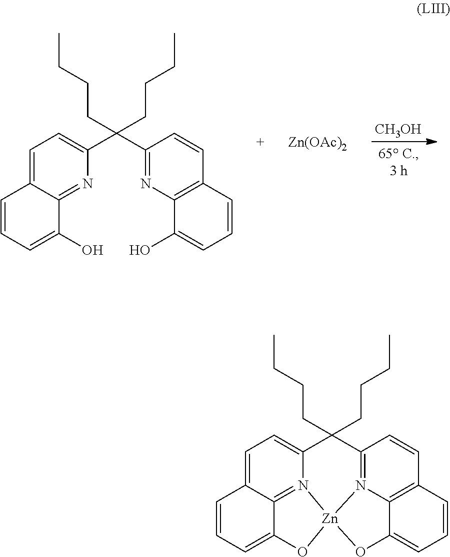

Yield: 647 mg (1.35 mmol, 89%), light-yellow powder,

HR-ESI/MS (CH₃OH): m/z calculated for [C₂₇H₂₈N₂O₂Zn+Na]⁺: 499.1334. found: 499.1344.

¹H-NMR (300.1 MHz, (CD₃)₂SO): δ (ppm)=0.75 (t, J=6.9 Hz, 6H, CH₃), 0.83-1.06 (m, 4H, CH₂Et), 1.23 (s, 4H, CH₂CH₃), 2.47 (s, 4H, CCH₂), 6.89 (d, J=8.0 Hz, 2H, H7), 6.78 (d, J=7.7 Hz, 2H, H5), 7.36 (t, J=7.9 Hz, 2H, H6), 7.88 (d, J=8.8 Hz, 2H, H3), 8.37 (d, J=8.9 Hz, 2H, H4).

¹³C{¹H}-NMR (75.5 MHz, (CD₃)₂SO): δ (ppm)=13.7 (CH₃), 22.3 (CH₂CH₃), 26.6 (CH₂Et), 40.3 (CCH₂), 51.9 (CCH₂), 108.4 (C5), 111.9 (C7), 120.5 (C3), 127.3 (C4a), 129.8 (C6), 138.8 (C4), 139.0 (C8a), 159.9 (C2), 162.4 (C8).

CHN analysis: calculated for C₂₇H₂₈N₂O₂Zn (477.93 g/mol): C, 67.85; H, 5.91; N, 5.86 wt %. found: C, 59.31; H, 5.83; N, 4.65 wt %.

### Example 1-1-1

### Synthesis of Polycyclobexene Carbonate with the Complex [Cr(Babhq)OC(O)CF₃] from Example 1-1 in the Presence of PPNCl

Polycyclohexene carbonate was prepared according to formula (LIV) by reacting cyclohexene oxide with CO₂. The letters given in the formula (LIV) refer here to the designation of the atoms for the signal assignment in the NMR spectra.

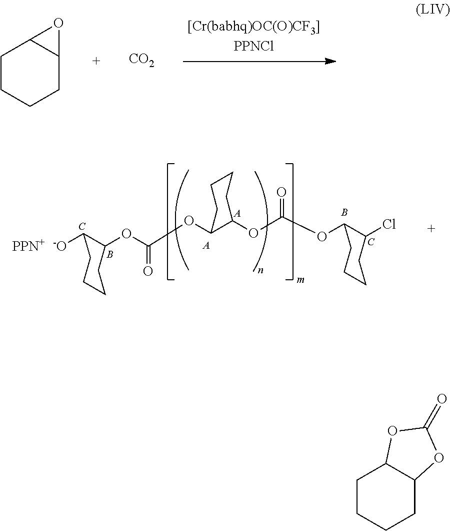

Cr(III) catalyst [Cr(babhq)OC(O)CF₃] (13.6 mg, 0.026 mmol), bis(triphenylphosphoranylidene)ammonium chloride (PPNCl, 34.8 mg, 0.061 mmol) and cyclohexene oxide (15.3 mL, 151 mmol) were charged in a 160 mL autoclave, equipped with a hollow shaft stirrer, and contacted with CO₂ (20 bar) at room temperature (molar ratio cyclohexene oxide/catalyst/PPNCl=4885/1/2.3). The reaction mixture was then heated to 100° C. (over a period of 10 min) and stirred at 700 rpm for 10 hours. The autoclave was then cooled to 15° C. with the aid of an ice bath and the remaining CO₂ excess pressure was slowly released. After opening the autoclave, an NMR sample was taken for quantitative analysis and was measured in CDCl₃.

The time-concentration profile measured by in situ IR spectroscopy is shown in FIG. 1.

Amount of unreacted cyclohexene oxide: 24.0%

Yield of cyclic cyclohexene carbonate: 0.8%

Yield of linear cyclohexene carbonate: 75.2%

A highly viscous material remained as residue which was dissolved in methylene chloride and transferred to a 250 mL round-bottom flask. After distillative removal of the volatile components on a rotary evaporator in a water bath (50° C.), the polymer was obtained as a pale orange powder.

A sample of the powder was dissolved in CDCl₃ and analyzed by ¹H-NMR spectroscopy.

Only the characteristic signals of the linear polycyclohexene carbonate formed by alternating incorporation of cyclohexene oxide and carbon dioxide into the polymer chain were observed.

¹H-NMR (pCHC, CDCl₃) δ (ppm)=4.51 (m, H^{A}), 4.28 (m, H^{B}), 3.41 (m, H^{C}).

Analysis of the product by GPC showed Mₙ=3831 g/mol and a PDI of 1.20.

### Example 1-1-2

### Synthesis of Polycyclohexene Carbonate with the Complex [Cr(Babhq)OC(O)CF₃] from Example 1-1 in the Presence of TBACl and 1,8-Octanediol

Polycyclohexene carbonate was prepared according to formula (LV) by reacting cyclohexene oxide with CO₂. The letters given in the formula (LV) refer here to the designation of the atoms for the signal assignment in the NMR spectra.

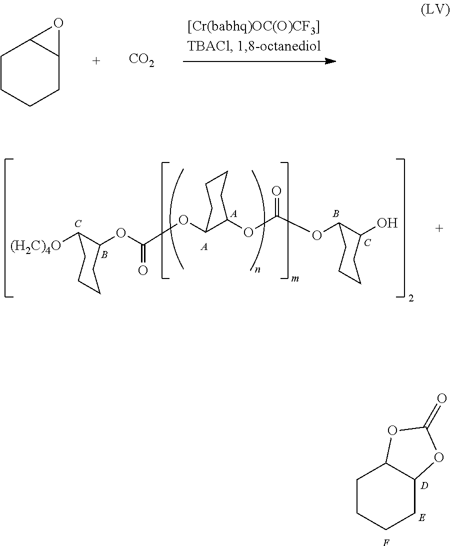

Cr(III) catalyst [Cr(babhq)OC(O)CF₃] (12.3 mg, 0.022 mmol), tetrabutylammonium chloride (TBACl, 26.0 mg, 0.094 mmol), 1,8-octanediol (121 mg, 0.828 mmol) and cyclohexene oxide (8.6 mL, 85 mmol) were charged in a 160 mL autoclave, equipped with a hollow shaft stirrer, and contacted with CO₂ (20 bar) at room temperature (molar ratio cyclohexene oxide/catalyst/TBACl=3542/1/3.9). The reaction mixture was then heated to 100° C. (over a period of 10 min) and stirred at 700 rpm for 6 hours. The autoclave was then cooled to 15° C. with the aid of an ice bath and the remaining CO₂ excess pressure was slowly released. After opening the autoclave, an NMR sample was taken for quantitative analysis and was measured in CDCl₃.

The time-concentration profile measured by in situ IR spectroscopy is shown in FIG. 1.

Amount of unreacted cyclohexene oxide: 10.9%

Yield of cyclic cyclohexene carbonate: 20.1%

Yield of linear cyclohexene carbonate: 69.0%

A highly viscous material remained as residue which was dissolved in methylene chloride and transferred to a 250 mL round-bottom flask. After distillative removal of the volatile components on a rotary evaporator in a water bath (50° C.), the polymer was obtained as a pale orange powder.

A sample of the powder was dissolved in CDCl₃ and analyzed by ¹H-NMR spectroscopy.

The characteristic signals of the linear polycyclohexene carbonate formed by non-alternating incorporation of cyclohexene oxide and carbon dioxide into the polymer chain, signals for 1,8-octanediol incorporated into the polymer chain and also the characteristic signals of the cyclic cyclohexene carbonate were observed.

¹H-NMR (CDCl₃) δ (ppm)=4.49 (b, H^{A}, linear polycyclohexene carbonate units), 4.34 (b, H^{A}, linear polycyclohexene ether units), 4.11 (b, H^{B}), 3.84-3.81 (m, H^{D}), 3.24 (b, H^{C}), 1.95 (m, H^{E}), 1.86-1.09 (m, H^{F} and CH₂ of 1,8-octanediol).

### Example 1-1-3

### Synthesis of Cyclic Propylene Carbonate with the Complex [Cr(Babhq)OC(O)CF₃] from Example 1-1 in the Presence of PPNCl

Cyclic propylene carbonate was prepared according to formula (LVI) by reacting propylene oxide with CO₂. The letters given in the formula (LVI) refer here to the designation of the atoms for the signal assignment in the NMR spectra.

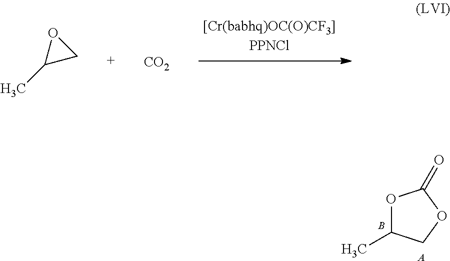

Cr(III) catalyst [Cr(babhq)OC(O)CF₃] (11 mg, 0.021 mmol), bis(triphenylphosphoranylidene)ammonium chloride (PPNCl, 29.0 mg, 0.050 mmol) and propylene oxide (12.0 mL, 171 mmol) were charged in a 160 mL autoclave, equipped with a hollow shaft stirrer, and contacted with CO₂ (20 bar) at room temperature (molar ratio propylene oxide/catalyst/PPNCl=8143/1/2.4). The reaction mixture was then heated to 100° C. (over a period of 10 min) and stirred at 700 rpm for 3.5 hours. The autoclave was then cooled to 15° C. with the aid of an ice bath and the remaining CO₂ excess pressure was slowly released. After opening the autoclave, an NMR sample was taken for quantitative analysis and was measured in CDCl₃.

Amount of unreacted propylene oxide: 13.0%

Yield of cyclic propylene carbonate: 87.0%

The residue remaining was a solution which was yellow-orange due to the catalyst and which was transferred to a round-bottom flask. After distillative removal of the volatile components on a rotary evaporator in a water bath (50° C.), the cyclic propylene carbonate was obtained as a yellow-orange liquid.

A sample was dissolved in CDCl₃ and analyzed by ¹H-NMR spectroscopy.

Only the characteristic signals of the cyclic propylene carbonate were observed.

¹H-NMR (400 MHz, CDCl₃) δ (ppm)=4.83-4.90 (1H, m, H^{B}), 4.57 (1H, dd, H^{A}), 4.04 (dd, H^{A}), 1.49 (3H, d, CH₃).

### Example 1-1-4

### Synthesis of Cyclic Propylene Carbonate with the Complex [Cr(Babhq)OC(O)CF₃] from Example 1-1 in the Presence of TBABr and 1,8-Octanediol

Cyclic propylene carbonate was prepared according to formula (LVII) by reacting propylene oxide with CO₂. The letters given in the formula (LVII) refer here to the designation of the atoms for the signal assignment in the NMR spectra.

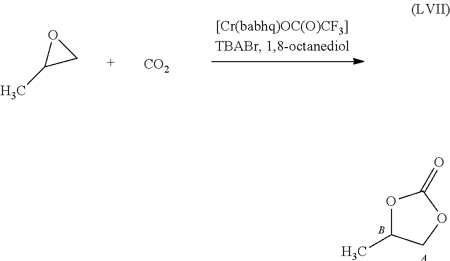

Cr(III) catalyst [Cr(babhq)OC(O)CF₃] (11.8 mg, 0.023 mmol), tetrabutylammonium bromide (TBABr, 24.8 mg, 0.077 mmol), 1,8-octanediol (126 mg, 0.862 mmol) and propylene oxide (7.0 mL, 99 mmol) were charged in a 160 mL autoclave, equipped with a hollow shaft stirrer, and contacted with CO₂ (20 bar) at room temperature (molar ratio propylene oxide/catalyst/TBABr=4304/1/33). The reaction mixture was then heated to 60° C. (over a period of 5 min) and stirred at 700 rpm for 4 hours. The autoclave was then cooled to 15° C. with the aid of an ice bath and the remaining CO₂ excess pressure was slowly released. After opening the autoclave, an NMR sample was taken for quantitative analysis and was measured in CDCl₃.

Amount of unreacted propylene oxide: 38.0%

Yield of cyclic propylene carbonate: 62.0%

The residue remaining was a solution which was yellow-orange due to the catalyst and which was transferred to a round-bottom flask. After distillative removal of the volatile components on a rotary evaporator in a water bath (50° C.), the cyclic propylene carbonate was obtained as a yellow-orange liquid.

A sample was dissolved in CDCl₃ and analyzed by ¹H-NMR spectroscopy.

Only the characteristic signals of the cyclic propylene carbonate were observed.

¹H-NMR (400 MHz, CDCl₃) δ (ppm)=4.83-4.90 (1H, m, H⁸), 4.57 (1H, dd, H^{A}), 4.04 (dd, H^{A}), 1.49 (3H, d, CH₃).

### Example 1-1-5

### Synthesis of Cyclic Propylene Carbonate with the Complex [Cr(Babhq)OC(O)CF₃] from Example 1-1 in the Presence of TBACl and 1,8-Octanediol

Cyclic propylene carbonate was prepared according to formula (LVIII) by reacting propylene oxide with CO₂. The letters given in the formula (LVIII) refer here to the designation of the atoms for the signal assignment in the NMR spectra.

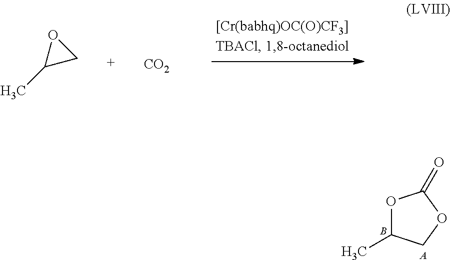

Cr(III) catalyst [Cr(babhq)OC(O)CF₃] (10.6 mg, 0.020 mmol), tetrabutylammonium chloride (TBACl, 24.0 mg, 0.086 mmol), 1,8-octanediol (126 mg, 0.862 mmol) and propylene oxide (9.4 mL, 133 mmol) were charged in a 160 mL autoclave, equipped with a hollow shaft stirrer, and contacted with CO₂ (20 bar) at room temperature (molar ratio propylene oxide/catalyst/TBACl=4304/1/4.1). The reaction mixture was then heated to 100° C. (over a period of 10 min) and stirred at 700 rpm for 16 hours. The autoclave was then cooled to 15° C. with the aid of an ice bath and the remaining CO₂ excess pressure was slowly released. After opening the autoclave, an NMR sample was taken for quantitative analysis and was measured in CDCl₃.

Amount of unreacted propylene oxide: <1%

Yield of cyclic propylene carbonate: >99%

The residue remaining was a solution which was yellow-orange due to the catalyst and which was transferred to a round-bottom flask. After distillative removal of the volatile components on a rotary evaporator in a water bath (50° C.), the cyclic propylene carbonate was obtained as a yellow-orange liquid.

A sample was dissolved in CDCl₃ and analyzed by ¹H-NMR spectroscopy.

Only the characteristic signals of the cyclic propylene carbonate were observed.

¹H-NMR (400 MHz, CDCl₃) δ (ppm)=4.83-4.90 (1H, m, H^{B}), 4.57 (1H, dd, H^{A}), 4.04 (dd, H^{A}), 1.49 (3H, d, CH₃).

### Example 1-2-1

### Synthesis of Cyclic Propylene Carbonate with the Complex [Co(Babhq)OC(O)CF₃] from Example 1-2 in the Presence of TBABr

Cyclic propylene carbonate was prepared according to formula (LIX) by reacting propylene oxide with CO₂. The letters given in the formula (LIX) refer here to the designation of the atoms for the signal assignment in the NMR spectra.

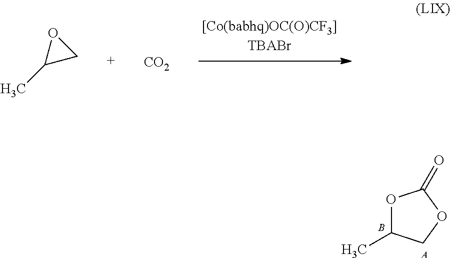

Co(III) catalyst [Co(babhq)OC(O)CF₃] (12.0 mg, 0.023 mmol), tetrabutylammonium bromide (TBABr, 13.0 mg, 0.040 mmol) and propylene oxide (7.2 mL, 102 mmol) were charged in a 160 mL autoclave, equipped with a hollow shaft stirrer, and contacted with CO₂ (20 bar) at room temperature (molar ratio propylene oxide/catalyst/TBABr=4435/1/1.7). The reaction mixture was then heated to 60° C. (over a period of 5 min) and stirred at 700 rpm for 2.5 hours. The autoclave was then cooled to 15° C. with the aid of an ice bath and the remaining CO₂ excess pressure was slowly released. After opening the autoclave, an NMR sample was taken for quantitative analysis and was measured in CDCl₃.

Amount of unreacted propylene oxide: 62.0%

Yield of cyclic propylene carbonate: 38.0%

The residue remaining was a solution which was yellow-orange due to the catalyst and which was transferred to a round-bottom flask. After distillative removal of the volatile components on a rotary evaporator in a water bath (50° C.), the cyclic propylene carbonate was obtained as a yellow-orange liquid.

A sample was dissolved in CDCl₃ and analyzed by ¹H-NMR spectroscopy.

Only the characteristic signals of the cyclic propylene carbonate were observed.

¹H-NMR (400 MHz, CDCl₃) δ (ppm)=4.83-4.90 (1H, m, H^{B}), 4.57 (1H, dd, H^{A}), 4.04 (dd, H^{A}), 1.49 (3H, d, CH₃).

### Example 2-1-1

### Synthesis of Cyclic Propylene Carbonate with the Complex [Co(Bpphen)OC(O)CF₃] from Example 1-2 in the Presence of PPNCl and 1-Octanol

Cyclic propylene carbonate was prepared according to formula (LX) by reacting propylene oxide with CO₂. The letters given in the formula (LX) refer here to the designation of the atoms for the signal assignment in the NMR spectra.

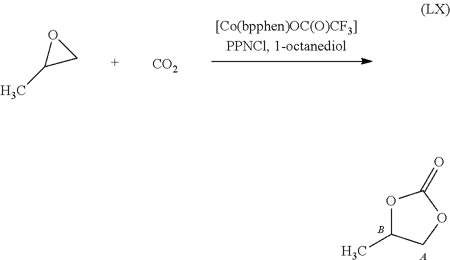

Co(III) catalyst [Co(bpphen)OC(O)CF₃] (10.7 mg, 0.020 mmol), bis(triphenylphosphoranylidene)ammonium chloride (PPNCl, 23.0 mg, 0.040 mmol), 1-octanol (0.31 mL, 2 mmol) and propylene oxide (7.0 mL, 99 mmol) were charged in a 160 mL autoclave, equipped with a hollow shaft stirrer, and contacted with CO₂ (20 bar) at room temperature (molar ratio propylene oxide/catalyst/TBABr=4950/1/2.0). The reaction mixture was then heated to 60° C. (over a period of 5 min) and stirred at 700 rpm for 2.5 hours. The autoclave was then cooled to 15° C. with the aid of an ice bath and the remaining CO₂ excess pressure was slowly released. After opening the autoclave, an NMR sample was taken for quantitative analysis and was measured in CDCl₃.

Amount of unreacted propylene oxide: 95.5%

Yield of cyclic propylene carbonate: 4.5%

The residue remaining was a solution which was yellow-orange due to the catalyst and which was transferred to a round-bottom flask. After distillative removal of the volatile components on a rotary evaporator in a water bath (50° C.), the cyclic propylene carbonate was obtained as a yellow-orange liquid.

A sample was dissolved in CDCl₃ and analyzed by ¹H-NMR spectroscopy.

Only the characteristic signals of the cyclic propylene carbonate were observed.

¹H-NMR (400 MHz, CDCl₃) δ (ppm)=4.83-4.90 (1H, m, H^{B}), 4.57 (1H, dd, H^{A}), 4.04 (dd, H^{A}), 1.49 (3H, d. CH₃).

### Example 3-1-1

### Synthesis of Cyclic Cyclohexene Carbonate with the Complex [Zn(Nbhq)] from Example 3-1 in the Presence of PPNCl and TBABr

Cyclic cyclohexene carbonate was prepared according to formula (LXI) by reacting cyclohexene oxide with CO₂. The letters given in the formula (LXI) refer here to the designation of the atoms for the signal assignment in the NMR spectra.

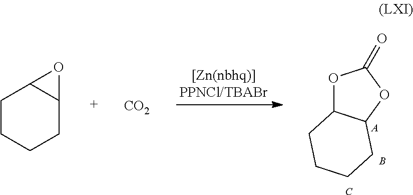

Zn(II) catalyst [Zn(nbhq)] (10.5 mg, 0.022 mmol), bis(triphenylphosphoranylidene)ammonium chloride (PPNCl, 30.0 mg, 0.052 mmol), tetrabutylammonium bromide (TBABr, 60 mg, 0.186 mmol) and cyclohexene oxide (13.0 mL, 128 mmol) were charged in a 160 mL autoclave, equipped with a hollow shaft stirrer, and contacted with CO₂ (20 bar) at room temperature (molar ratio cyclohexene oxide/catalyst/(PPNCl+TBABr)=6400/1/11.9). The reaction mixture was then heated to 100° C. (over a period of 10 min) and stirred at 700 rpm for 3.5 hours. The autoclave was then cooled to 15° C. with the aid of an ice bath and the remaining CO₂ excess pressure was slowly released. After opening the autoclave, an NMR sample was taken for quantitative analysis and was measured in CDCl₃.

Amount of unreacted cyclohexene oxide: 37.0%

Yield of cyclic cyclohexene carbonate: 63.0%

Yield of linear cyclohexene carbonate: 0%

The residue remaining was a liquid which was yellow due to the catalyst and which was transferred to a round-bottom flask. After distillative removal of the volatile components on a rotary evaporator in a water bath (50° C.), the cyclic cyclohexene carbonate was obtained as a yellow-orange solution.

A sample was dissolved in CDCl₃ and analyzed by ¹H-NMR spectroscopy.

Only the characteristic signals of the cyclic cyclohexene carbonate were observed.

¹H-NMR (400 MHz, CDCl₃) δ (ppm)=3.81 (2H, dd, H^{A}), 1.98 (4H, m, Ha), 0.97-1.01 (4H, m, H^{C}).

### Example 3-1-2

### Synthesis of Cyclic Propylene Carbonate with the Complex [Zn(Nbhq)] from Example 3-1 in the Presence of TBABr

Cyclic propylene carbonate was prepared according to formula (LXII) by reacting propylene oxide with CO₂. The letters given in the formula (LXII) refer here to the designation of the atoms for the signal assignment in the NMR spectra.

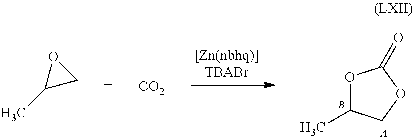

Zn(II) catalyst [Zn(nbhq)] (12.6 mg, 0.026 mmol), tetrabutylammonium bromide (TBABr, 26.0 mg, 0.081 mmol) and propylene oxide (9.0 mL, 127 mmol) were charged in a 160 mL autoclave, equipped with a hollow shaft stirrer, and contacted with CO₂ (20 bar) at room temperature (molar ratio propylene oxide/catalyst/TBABr=4885/1/3.1). The reaction mixture was then heated to 60° C. (over a period of 5 min) and stirred at 700 rpm for 12 hours. The autoclave was then cooled to 15° C. with the aid of an ice bath and the remaining CO₂ excess pressure was slowly released. After opening the autoclave, an NMR sample was taken for quantitative analysis and was measured in CDCl₃.

Amount of unreacted propylene oxide: 24.0%

Yield of cyclic propylene carbonate: 76.0%

Yield of linear propylene carbonate: 0%

The residue remaining was a liquid which was yellow due to the catalyst and which was transferred to a round-bottom flask. After distillative removal of the volatile components on a rotary evaporator in a water bath (50° C.), the cyclic propylene carbonate was obtained as a yellow-orange solution.

A sample was dissolved in CDCl₃ and analyzed by ¹H-NMR spectroscopy.

Only the characteristic signals of the cyclic propylene carbonate were observed.

¹H-NMR (400 MHz, CDCl₃) δ (ppm)=4.83-4.90 (1H, m, H^{B}), 4.57 (1H, dd, H^{A}), 4.04 (dd, H^{A}), 1.49 (3H, d, CH₃).

### Comparison of Examples 1-1-1 to 3-1-2

An overview of the results obtained is shown in the following table.

TABLE 1
Overview of the results of examples 1-1-1 to 3-1-2 (key to abbreviations:
PPNCl: bis(triphenylphosphoranylidene)ammonium chloride; TBACl: tetrabutylammonium
chloride; TBABr: tetrabutylammonium bromide; 1,8-OL: 1,8-octanediol;
PO: propylene oxide; CHO: cyclohexene oxide)

| | Complex used | Co-catalyst/ | | Unreacted | Yield of cyclic | Yield of linear |
|---|---|---|---|---|---|---|
| Example | as catalyst | starter | Substrate | epoxide | carbonate ester | carbonate ester |
| 1-1-1 | [Cr(babhq)OC(O)CF₃] | PPNCl/— | CHO | 24.0% | 0.8% | 75.2% |
| 1-1-2 | [Cr(babhq)OC(O)CF₃] | TBACl/1,8-OL | CHO | 10.9% | 20.1% | 69.0% |
| 1-1-3 | [Cr(babhq)OC(O)CF₃] | PPNCl/— | PO | 13.0% | 87.0% | 0% |
| 1-1-4 | [Cr(babhq)OC(O)CF₃] | TBABr/1,8-OL | PO | 38.0% | 62.0% | 0% |
| 1-1-5 | [Cr(babhq)OC(O)CF₃] | TBACl/1,8-OL | PO | <1% | >99% | 0% |
| 1-2-1 | [Co(babhq)OC(O)CF₃] | TBABr/— | PO | 62.0% | 38.0% | 0% |
| 2-1-1 | [Co(bpphen)OC(O)CF₃] | PPNCl/1-octanol | PO | 95.5 | 4.5 | 0% |
| 3-1-1 | [Zn(nbhq)] | PPNCl/TBABr | CHO | 37.0% | 63.0% | 0% |
| 3-1-2 | [Zn(nbhq)] | TBABr/— | PO | 24.0% | 76.0% | 0% |

The examples confirm that the complexes [Cr(babhq)OC(O)CF₃], [Co(babhq)OC(O)CF₃] and [Zn(nbhq)], used as catalyst for the formation of linear and cyclic carbonate esters from the cyclohexene oxide and propylene oxide compounds used as epoxides and carbon dioxide, are active.

### FIGURES

FIG. 1 describes the time-concentration profile for the reaction of cyclohexene oxide and carbon dioxide with [Cr(babhq)OC(O)CF₃] described in Example 1-1-1. The time in minutes is plotted on the x-axis and the mole fraction in mol/mol is plotted on the y-axis. The downward curve describes the concentration of cyclohexene oxide and the rising curve the concentration of linear cyclohexene carbonate. The concentration of the cyclic cyclohexene carbonate was below the limit of detection. A solid line, largely concealed by the data points, represents a mathematical modeling of the measured data according to a first-order reaction.

FIG. 2 describes the time-concentration profile for the reaction of propylene oxide and carbon dioxide with [Cr(babhq)OC(O)CF₃] described in Example 1-1-3. The time in minutes is plotted on the x-axis and the mole fraction in mol/mol is plotted on the y-axis. The downward curve describes the concentration of propylene oxide and the rising curve the concentration of cyclic propylene carbonate. The solid line represents a mathematical modeling of the measured data according to a second-order reaction.

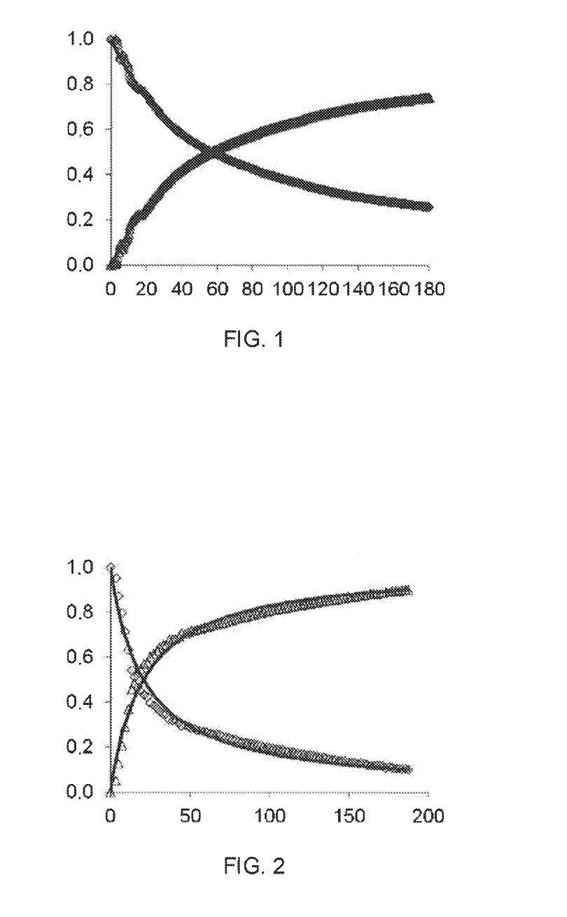

1.-11. (canceled)

12. A method for preparing linear and/or cyclic carbonate esters comprising the step of reacting an epoxide with carbon dioxide in the presence of a catalyst,
wherein
the catalyst comprises a complex of a metal M or M-A with a ligand L, the metal M being selected from the group of Zn(II), Cr(II), Mn(II), Mg(II), Fe(II), Co(II), Cr(III)-A, Mn(III)-A, Fe(III)-A and/or Co(m)-A,
A is halide, carboxylate, phenolate, sulfonate, phosphonate, alkyl, alkoxy or amido
and the ligand L has the following structure (Ia) or (Ib), wherein one or both of the OH groups shown in (Ia) and/or (Ib) can also be deprotonated: 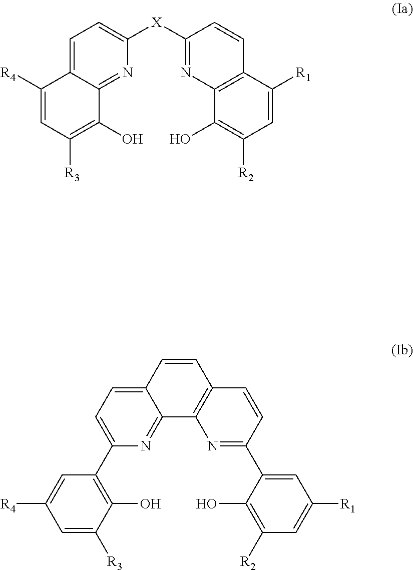
where R1, R2, R3 and R4 are each independently hydrogen, a C₁-C₂₂-alkyl residue, a C₅-C₁₂-cycloalkyl residue, a C₇-C₁₄-aralkyl or -alkylaryl residue or a C₆-C₁₄-aryl residue and X is a bridge of the formula (II) or (III): 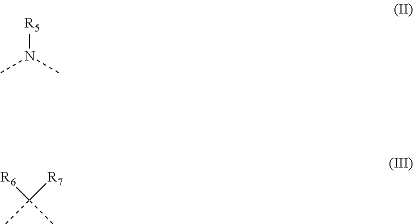
where R5 is a C₁-C₂₂-alkyl residue, a C₅-C₁₂-cycloalkyl residue, a C₇-C₁₄-aralkyl or -alkylaryl residue, a C6-C14-aryl residue, or an optionally alkyl-substituted pyridyl residue or pyridylmethyl residue and
R6 and R7 are each independently hydrogen, a C₁-C₂₂-alkyl residue, a C₅-C₁₂-cycloalkyl residue, a C₇-C₁₄-aralkyl or -alkylaryl residue, a C₆-C₁₄-aryl residue or a —COOR8 residue, where R8 is a C₁-C₈-alkyl residue.

13. The method as claimed in claim 12, wherein A is OCOCH₃, OCOCF₃, OSO₂CF₃, OSO₂C₆H₅CH₃, or halide.

14. The method as claimed in claim 12, wherein M is selected from the group of Zn(II), Cr(III)-A and/or Co(III)-A.

15. The method as claimed in claim 12, wherein the ligand L is selected from the group consisting of compounds of the formulae (IV) to (VII):

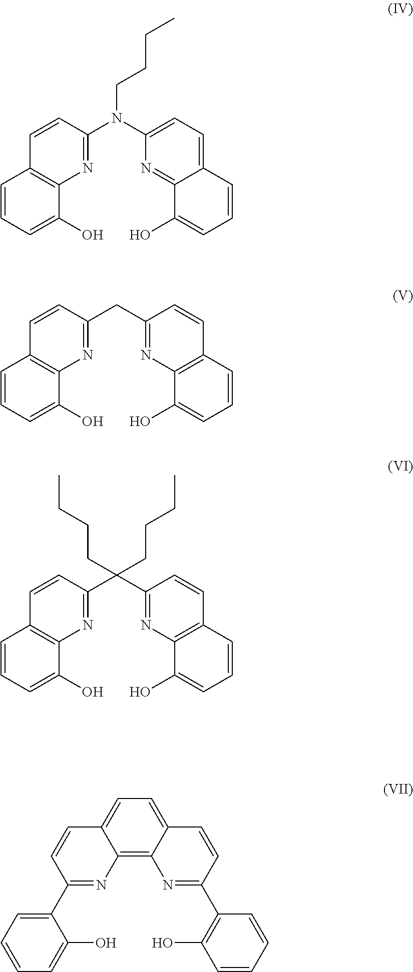 [SMILES: CCCCN(c1ccc2cccc(O)c2n1)c1ccc2cccc(O)c2n1 ; Oc1cccc2ccc(Cc3ccc4cccc(O)c4n3)nc12 ; CCCCC(CCCC)(c1ccc2cccc(O)c2n1)c1ccc2cccc(O)c2n1 ; Oc1ccccc1-c1ccc2ccc3ccc(-c4ccccc4O)nc3c2n1]

16. The method as claimed in claim 12, wherein the catalyst is selected from the group consisting of compounds of the formulae (VIII) to (XVIII):

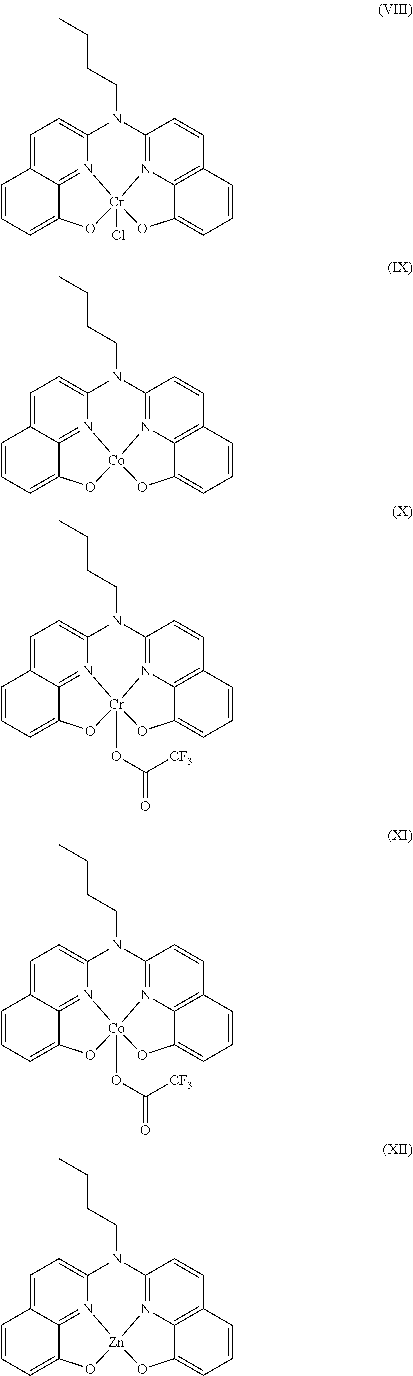

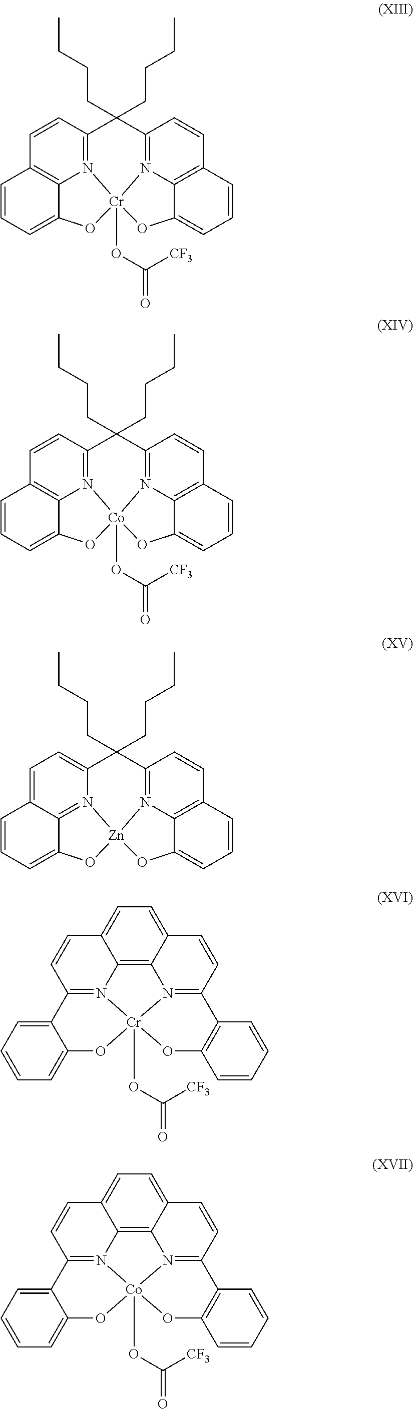

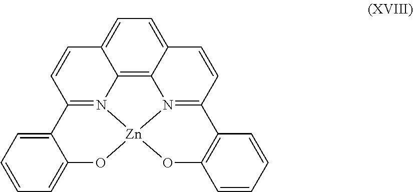

17. The method as claimed in claim 12, wherein the epoxide is ethylene oxide, propylene oxide, cyclohexene oxide or styrene oxide or any mixture comprising ethylene oxide, propylene oxide, cyclohexene oxide and styrene oxide.

18. The method as claimed in claim 12, wherein the reaction is conducted at a temperature of ≧60° C. to ≦120° C.

19. The method as claimed in claim 12, wherein the catalyst is heated to a temperature of >40° C. to ≦150° C. prior to contact with the epoxide.

20. The method as claimed in claim 12, further comprising utilizing a co-catalyst selected from the group consisting of ammonium salts, phosphonium salts, bis(triphenylphosphine)iminium salts, amidines, guanidines and DMAP in the reaction.

21. The method as claimed in claim 12, wherein the reaction is conducted in the presence of an alcohol having ≧1 to ≦8 OH groups.

22. A method comprising utilizing complexes of a metal M or M-A with a ligand L as catalysts for the reaction of an epoxide with carbon dioxide, wherein the metal M is present in an oxidation state of ≧0,
A is halide, carboxylate, phenolate, sulfonate, phosphonate, alkyl, alkoxy or amido
and the ligand L has the following structure (Ia) or (Ib), wherein one or both of the OH groups shown in (Ia) and/or (Ib) can also be deprotonated: 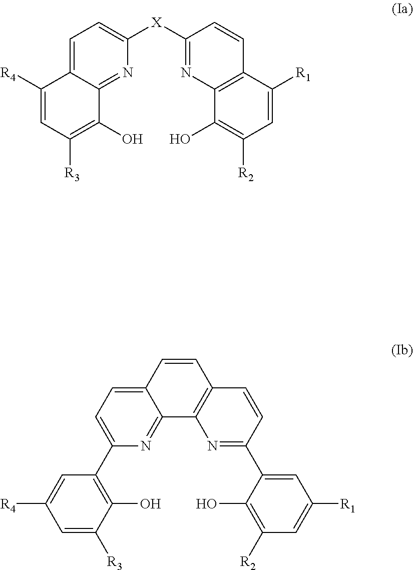
where R1, R2, R3 and R4 are each independently hydrogen, a C₁-C₂₂-alkyl residue, a C₅-C₁₂-cycloalkyl residue, a C₇-C₁₄-aralkyl or -alkylaryl residue or a C₆-C₁₄-aryl residue and X is a bridge of the formula (II) or (III): 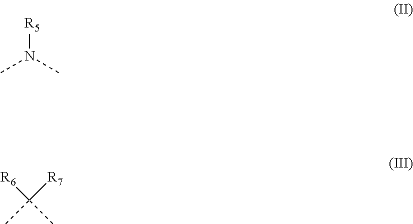
where R5 is a C₁-C₂₂-alkyl residue, a C₅-C₁₂-cycloalkyl residue, a C₇-C₁₄-aralkyl or -alkylaryl residue, a C₆-C₁₄-aryl residue, or an optionally alkyl-substituted pyridyl residue or pyridylmethyl residue and
R6 and R7 are each independently hydrogen, a C₁-C₂₂-alkyl residue, a C₈-C₁₂-cycloalkyl residue, a C₇-C₁₄-aralkyl or -alkylaryl residue, a C6-C₁₄-aryl residue or a —COOR8 residue, where R8 is a C₁-C₈-alkyl residue.
